(19) United States
(12) Patent Application Publication
(10) Pub. No.: US 2007/0238719 A1
(43) Pub. Date: Oct. 11, 2007

(54) Method for reducing the risk of or preventing infection due to surgical or invasive medical procedures

(76) Inventors: Scott J. Hopkins, Old Saybrook, CT (US); Robert E. Kessler, Branford, CT (US); Albert R. Collinson, Marlborough, MA (US); Joyce A. Sutcliffe, Branford, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/432,228

(22) Filed: May 10, 2006

Related U.S. Application Data
(60) Provisional application No. 60/715,099, filed on Sep. 8, 2005.
(60) Provisional application No. 60/715,079, filed on Sep. 8, 2005.
(60) Provisional application No. 60/712,459, filed on Aug. 29, 2005.
(60) Provisional application No. 60/712,311, filed on Aug. 29, 2005.
(60) Provisional application No. 60/702,349, filed on Jul. 25, 2005.
(60) Provisional application No. 60/681,398, filed on May 16, 2005.
(60) Provisional application No. 60/680,097, filed on May 12, 2005.
(60) Provisional application No. 60/679,512, filed on May 10, 2005.
(60) Provisional application No. 60/679,511, filed on May 10, 2005.
(60) Provisional application No. 60/679,475, filed on May 10, 2005.
(60) Provisional application No. 60/679,425, filed on May 10, 2005.

(51) Int. Cl. A61K 31/4709

(52) U.S. Cl. 514/210.21

(57) ABSTRACT

The present invention relates to methods for reducing the risk of infection due to surgical or invasive medical procedures. The present invention also relates to methods for preventing infection due to surgical or invasive medical procedures.

### RELATED APPLICATIONS

This application claims the benefit of and priority to U.S. patent application Ser. No. 60/715,099, filed Sep. 8, 2005, U.S. patent application Ser. No. 60/715,079, filed Sep. 8, 2005, U.S. patent application Ser. No. 60/712,459, filed Aug. 29, 2005, U.S. patent application Ser. No. 60/712,311, filed Aug. 29, 2005, U.S. patent application Ser. No. 60/702,349, filed Jul. 25, 2005, U.S. patent application Ser. No. 60/681,398, filed May 16, 2005, U.S. patent application Ser. No. 60/680,097, filed May 12, 2005, U.S. patent application Ser. No. 60/679,512, filed May 10, 2005, U.S. patent application Ser. No. 60/679,511, filed May 10, 2005, U.S. patent application Ser. No. 60/679,475, filed May 10, 2005, and U.S. patent application Ser. No. 60/679,425, filed May 10, 2005, the disclosures of which are incorporated by reference herein.

### FIELD OF THE INVENTION

The present invention relates to methods for reducing the risk of infection due to surgical or invasive medical procedures. The present invention also relates to methods for preventing infection due to surgical or invasive medical procedures.

### BACKGROUND

Infections at the site of surgery or other invasive medical procedures are a potentially serious risk for patients. See, e.g., D. W. Bratzler et al., “Antimicrobial Prophylaxis for Surgery: An Advisory Statement from the National Surgical Infection Prevention Project”, CID 2004:38 (15 June), pp. 1706-1715. Surgical site infections (also known as “SSIs”) are the second most common cause of nosocomial, i.e., hospital-acquired, infections. See, e.g., J. P. Burke, “Infection Control—A Problem for Patient Safety”, N. Engl. J. Med., 2003, 348, pp. 651-656 and “National Nosocomial Infections Surveillance (NNIS) report, data summary from October 1986-April 1996, issued May 1996: a report from the National Nosocomial Infections Surveillance (NNIS) System. Am. J. Infect. Control, 1996, 24, pp. 380-388. See, also, “National Nosocomial Infections Surveillance (NNIS) report, data summary from January 1992 through June 2004, issued October 2004: a report from the National Nosocomial Infections Surveillance (NNIS) System. Am. J. Infect. Control, vol. 32, no. 8, pp. 470-485 (December 2004). The incidence of these infections have been reported to range from about 2% to 5% of patients undergoing clean extraabdominal procedures, and up to about 20% of those undergoing intraabdominal procedures, with the total number of SSIs estimated at around 500,000 cases per year in the U.S. See, e.g., A. D. Auerbach, “Prevention of Surgical Site Infections.” In K. G. Shojana et al., eds., “Making health Care Safer: A Critical Analysis of Patient Safety Practices. Evidence report/technology assessment no.43. AHQR publication no. 01-E058. Rockville, Md.: Agency for Healthcare Research and Quality, 20 Jul. 2001, pp. 221-244 and E. S. Wong, “Surgical Site Infection”, in D. G. Mayhill, ed., “Hospital Epidemiology and Infection Control”, 2^{nd} ed., Philadelphia, Lippincott, Williams & Wilkins, 1999, pp. 189-210. Also, these infections are associated with a two-fold higher risk of death. See, e.g., K. G. Kirkland, et al., “The Impact of Surgical Site Infections in the 1990s: Attributable Mortality, Excess Length of Hospitalization, and Extra Costs”. Infect. Control. Hosp. Epidemiol., 1999, 20, pp. 725-730. See, also, W. J. Martone et al., “Incidence and Nature of Endemic and Epidemic Nosocomial Infections”, in J. V. Bennet et al., eds. “Hospital Infections”, 3^{rd} ed. New York: Little, Brown Medical Division, p. 577-96 (1992); C. S. Hollenbek et al., “Nonrandom Selection and the Attributable Cost of Surgical-Site Infections”, Infect. Control. Hosp. Epidemiol., 23, pp. 177-182 (2002); and E. N. Perencevich, et al., “Health and Economic Impact of Surgical Site Infections Diagnosed After Hospital Discharge”, Emerg. Infect. Dis., 9, pp. 196-203 (2003).

This problem of infection due to surgery or other invasive medical procedures is further compounded by the problem of resistance. Strains of microorganisms resistant to currently effective therapeutic agents continue to evolve. In fact, virtually every antibiotic agent developed for clinical use has ultimately encountered problems with the emergence of resistant bacteria. See, e.g., F. D. Lowry, “Antimicrobial Resistance: The Example of Staphylococcus aureus,” J. Clin. Invest., vol. 111, no. 9, pp. 1265-1273 (2003); and Gold, H. S. and Moellering, R. C., Jr., “Antimicrobial-Drug Resistance,” N. Engl. J. Med., vol. 335, no. 19, pp. 1445-1453 (1996).

Many of the antibiotic agents currently administered prophylactically have limitations in terms of adequate potency, cumbersome administration regimens, potential side-effects, and microbial resistance development. Consequently, there is a need for developing effective methods for reducing the risk of or preventing microbial infections due to surgical or other invasive procedures.

### SUMMARY O F THE INVENTION

The invention relates to a method of reducing the risk of a microbial infection in a patient having a surgical or invasive medical procedure by administering a prophylactically effective amount of an antimicrobial compound to the patient prior to the surgical or invasive procedure.

The invention also relates to a method of preventing a microbial infection in a patient having a surgical or invasive medical procedure by administering a prophylactically effective amount of an antimicrobial compound to the patient prior to the surgical or invasive procedure.

Additionally, the invention relates to a method of peri-operative prophylaxis in a patient in need thereof by administering a prophylactically effective amount of an antimicrobial compound to the patient prior to the patient undergoing a surgical or invasive medical procedure.

In one aspect, the invention relates to a composition for reducing the risk of a microbial infection in a patient having a surgical or invasive medical procedure, were the composition includes a prophylactically effective amount of an antimicrobial compound.

The invention also relates to a composition for preventing a microbial infection in a patient a having a surgical or invasive medical procedure, where the composition includes a prophylactically effective amount of an antimicrobial compound.

The invention also relates to a composition for peri-operative prophylaxis, where the composition includes a prophylactically effective amount of an antimicrobial compound.

Further, the invention includes the use of an antimicrobial compound in the manufacture of a composition for reducing the risk of a microbial infection in a patient having a surgical or invasive medical procedure, where the antimicrobial composition is administered prophylactically, in an effective amount, to a patient prior to the patient undergoing a surgical or invasive medical procedure.

The invention also relates to the use of an antimicrobial compound in the manufacture of a composition for preventing a microbial infection in a patient having a surgical or invasive medical procedure, where the antimicrobial composition is administered prophylactically, in an effective amount, to a patient prior to the patient undergoing a surgical or invasive medical procedure.

The invention relates, in part, to the use of an antimicrobial compound in the manufacture of a composition for peri-operative prophylaxis in a patient in need thereof, where the antimicrobial composition is administered prophylactically, in an effective amount, to a patient prior to the patient undergoing a surgical or invasive medical procedure.

In the methods, compositions, and uses of the invention, the microbial infection is, for example, a bacterial infection. For example, the microbial infection is a viral infection. Or, for example, the microbial infection is a fungal infection.

Examples of microbial infections include a skin infection, an abdominal infection, a urinary tract infection, bacteremia, septicemia, endocarditis, an infection associated with an atrio-ventricular shunt, a vascular access infection, meningitis, a peritoneal infection, a bone infection, a deep sternal wound infection, a joint infection, an infection associated with a catheter, an infection associated with a stent, an infection associated with a prosthetic device, a Gram-negative bacterial infection, a methicillin-resistant Staphylococcus aureus infection, a vancomycin-resistant enterococcal infection, a Bacteriodes infection, and a linezolid-resistant infection.

The compounds, compositions and methods of the invention are useful for administration to a mammal. For example, the patient can be a human.

In one example, an antimicrobial compound or composition is administered intravenously to the patient.

In another example, an antimicrobial compound or composition is administered orally to the patient.

In another example, an antimicrobial compound or composition is administered subcutaneously to the patient.

In another example, an antimicrobial compound or composition is administered parenteraly to the patient.

In another example, an antimicrobial compound or composition is administered intramuscularly to the patient.

The antimicrobial compound or composition can be administered to a patient before a surgical or invasive procedure. For example, the compound or composition is administered about 24 hours prior to a surgical or invasive procedure. In one example, the compound or composition is administered about 20 hours prior to the surgical or invasive procedure. In another example, the compound or composition is administered about 16 hours prior to the surgical or invasive procedure. In another example, the compound or composition is administered about 12 hours prior to the surgical or invasive procedure. In another example, the compound or composition is administered about 10 hours prior to the surgical or invasive procedure. In another example, the compound or composition is administered about 8 hours prior to the surgical or invasive procedure. In another example, the compound or composition is administered about 6 hours prior to the surgical or invasive procedure. In another example, the compound or composition is administered about 4 hours prior to the surgical or invasive procedure. In another example, the compound or composition is administered about 2 hours prior to the surgical or invasive procedure. In another example, the compound or composition is administered about 1 hour prior to the surgical or invasive procedure. In another example, the compound or composition is administered about 30 minutes prior to the surgical or invasive procedure.

The antimicrobial compound or composition can be administered to a patient during a surgical or invasive procedure.

The antimicrobial compound or composition can be administered to a patient up to 24 hours before, and, also, during a surgical or invasive procedure.

The antimicrobial compound or composition can be, for example, a compound that binds to or modulates ribosomal RNA. For example, the compound or composition can bind to or modulate bacterial ribosomal RNA.

The antimicrobial compound or composition can be, for example, a compound that binds to or modulates the large ribosomal subunit. For example, the compound or composition can bind to or modulate the large ribosomal subunit of a bacterial organism.

The antimicrobial compound or composition can be, for example, a compound that binds to or modulates a DNA topoisomerase. For example, the antimicrobial compound or composition can bind to or modulate a bacterial DNA topoisomerase. For example, the antimicrobial compound or composition can bind to or modulate bacterial topoisomerase IV.

The antimicrobial compound or composition can be, for example, a compound that binds to or modulates a bacterial DNA gyrase.

For example, the antimicrobial agent can be selected from macrolides, ketolides, streptogramin As, streptogramin Bs, chloramphenicol and chloramphenicol derivatives, fluorfenicol and fluorfenicol derivatives, glycopeptides, pleuromutilins, aminoglycosides, beta-lactams and carbapenems (including carbapenems with a 7-acylated imidazo[5-1,b]thiazole-2-yl group directly attached to the carbapenem moiety of the C-2 position), cephalosporins, lincosamides, quinolones, fluoroquinolones, pyridonecarboxylic acid derivatives, benzoheterocyclic compounds, aminomethylcycline compounds, dalbavancin, daptomycin, garenoxacin, gatifloxacin, gemifloxacin, levofloxacin, moxifloxacin, oritavancin, oxazolidinones (e.g., linezolid and other oxazolidinones), televancin, DK-507k [also known as, (−)-7-[(7S)-7-amino-5-azaspiro[2.4]heptan-5-yl]-6-fluoro-1-[(1R, 2S)-2-fluoro-1-cyclopropyl]-1,4-dihydro-8-methoxy-4-oxo-3-quinolinecarboxylic acid monohydrochloride monohydrate], and mixtures thereof.

The antimicrobial agent can be, for example, a pyridonecarboxylic acid derivative.

The pyridonecarboxylic acid derivative can be, for example, a compound according to the formula for Pyridonecarboxylic Acid Derivative 1:

Pyridonecarboxylic Acid Derivative 1

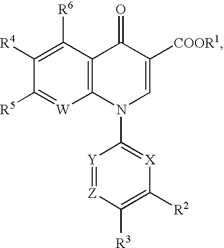

wherein R¹ represents a hydrogen atom or a carboxyl protective group; R² represents a hydroxyl group, a lower alkoxy group, or a substituted or unsubstituted amino group; R³ represents a hydrogen atom or a halogen atom; R⁴ represents a hydrogen atom or a halogen atom; R ⁵represents a halogen atom or an optionally substituted saturated cyclic amino group; R⁶ represents a hydrogen atom, a halogen atom, a nitro group, or an optionally protected amino group; X, Y and Z may be the same or different and respectively represent a nitrogen atom, CH or CR⁷ (wherein R⁷represents a lower alkyl group, a halogen atom, or a cyano group), provided that at least one of X, Y and Z represents a nitrogen atom, and W represents a nitrogen atom or CR⁸ (wherein R⁸ represents a hydrogen atom, a halogen atom, or a lower alkyl group), or a pharmaceutically acceptable salt, ester, or prodrug thereof, provided that when R¹ is hydrogen, R² is an amino group, R³ and R⁴ are each a fluorine atom, R⁶ is hydrogen, X is nitrogen, Y is CR⁷ (wherein R⁷ is fluorine), Z is CH, and W is CR⁸ (wherein R⁸ is chlorine), then R⁵ is not a 3-hydroxyazetidine-1-yl group.

The pyridonecarboxylic acid derivative can be, for example:

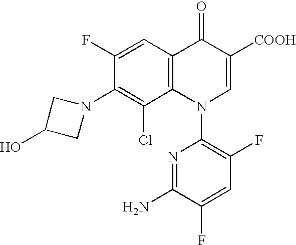

or a pharmaceutically acceptable salt, ester, or prodrug thereof.

The antimicrobial agent can be a D-glucitol l-(6-amino-3,5-difluoro-2-pyridinyl)-8-chloro-6-fluoro-1,4-dihydro-7-(3-hydroxy-1-azetidinyl)-4-oxo-3-quinolinecarboxylate (salt). For example, the antimicrobial compound or composition can be crystalline D-glucitol 1-(6-amino-3,5-difluoro-2-pyridinyl)-8-chloro-6-fluoro-1,4-dihydro-7-(3-hydroxy-1-azetidinyl)-4-oxo-3-quinolinecarboxylate (salt) characterized, when measured at about 25° C. with Cu-Ka radiation, by the powder diffraction pattern shown in FIG. 1. For example, the antimicrobial compound or composition can be D-glucitol 1-(6-amino-3,5-difluoro-2-pyridinyl)-8-chloro-6-fluoro-1,4-dihydro-7-(3-hydroxy-1-azetidinyl)-4-oxo-3-uinolinecarboxylate trihydrate (salt). For example, the antimicrobial compound or composition can be crystalline D-glucitol 1-(6-amino-3,5-difluoro-2-pyridinyl)-8-chloro-6-fluoro-1,4-dihydro-7-(3-ydroxy-1-azetidinyl)-4-oxo-3-quinolinecarboxylate trihydrate (salt) characterized, when measured at about 25° C. with Cu-Ka radiation, by the powder diffraction pattern shown in FIG. 2.

The antimicrobial compound or composition can be a benzoheterocyclic compound. For example, the antimicrobial compound or composition can be a benzoheterocyclic compound corresponding to Benzoheterocyclic Compound I:

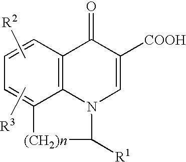

wherein R¹ represents a hydrogen atom or a lower alkyl group; R² represents a hydrogen atom or a halogen atom; R³ represents a 1-pyrrolidinyl group which may be substituted with a hydroxymethyl group, a 1,2,5,6-tetrahydro-1-pyridyl group, or a group of the formula:

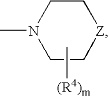

where R⁴ represents a hydrogen atom, a lower alkyl group, a lower alkoxy group, a hydroxy group, a phenyl-lower alkyl group, a lower alkanoyloxy group, an amino group which may be substituted with a lower alkyl group or a lower alkanoyl group, an oxo group, or a carbamoyl group; Z represents an oxygen atom, a sulfur atom or a methylene group; m is 1 or 2; and n is an integer of 1 or 2; or a pharmaceutically acceptable salt ester or prodrug thereof.

The antimicrobial agent can be a 9-fluoro-8-(4-hydroxy-1-piperidyl)-5-methyl-6,7-dihydro-1-oxo-1H,5H-benzo[i,j]quinolizine-2-carboxylic acid, or a pharmaceutically acceptable salt, ester, or prodrug thereof. For example, the antimicrobial agent can be S-(−)-9-fluoro-6,7-dihydro-8-(4-hydroxypiperidin-1-yl)-5-methyl-1-oxo-1H,5H-benzo[i,j]quinolizine-2-carboxylic acid or a pharmaceutically acceptable salt, ester, or prodrug thereof.

The antimicrobial agent can be S-(−)-9-fluoro-6,7-dihydro-8-(4-hydroxypiperidin-1-yl)-5-methyl-1-oxo-1H,5H-benzo[i,j]quinolizine-2-carboxylic acid arginine salt.

For example, the antimicrobial agent can be S-(−)-9-fluoro-6,7-dihydro-8-(4-hydroxypiperidin-1-yl)-5-methyl-1-oxo-1H,5H-benzo[i,j]quinolizine-2-carboxylic acid arginine salt having the following X-ray diffraction data: (2θ): 10.16, 11.78, 12,52, 16.00, 18.94, 19.66, 20.36, 21.28, 21.92, 22.52, 24.74, 25.28, 30.74.

For example, the antimicrobial agent can be S-(−)-9-fluoro-6,7-dihydro-8-(4-hydroxypiperidin-1-yl)-5-methyl-1-oxo-1 H,5H-benzo[i,j]quinolizine-2-carboxylic acid arginine salt having the following X-ray diffraction data: (2θ): 18.28, 18.8, 19.8, 20.12, 20.62, 21.10, 21.44, 21.88, 22.6, 23.02.

For example, the antimicrobial agent can be S-(−)-9-fluoro-6,7-dihydro-8-(4-hydroxypiperidin-1-yl)-5-methyl-1-oxo-1H,5H-benzo[i,j]quinolizine-2-carboxylic acid arginine salt having the following X-ray diffraction data: (2θ): 14.02±0.2, 14.82±0.2, 19.28±0.2, 22.12±0.2, 22.96±0.2, 23.46±0.2, 28.36±0.2.

The antimicrobial agent can be, for example, a beta-lactam compound.

For example, the compound or composition can be a carbapenem. In one embodiment, the compound or composition is a carbapenem with a 7-acylated imidazo[5-1,b]thiazole-2-yl group directly attached to the carbapenem moiety of the C-2 position.

The beta-lactam compound or composition can be a compound corresponding to the Beta-Lactam I:

Beta-Lactam I

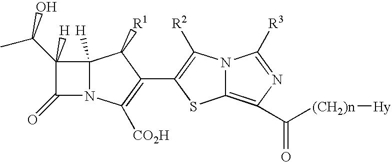

wherein R¹ represents a hydrogen atom or methyl, R² and R³, which may be the same or different, each represent a hydrogen atom; a halogen atom; lower alkyl optionally substituted by a halogen atom, cyano, hydroxyl, carbamoyl, amino, formylamino, lower alkylcarbonylamino, aminosulfonylamino, lower alkylthio, lower alkoxy, lower cycloalkyl, N,N-di-lower alkylamino, or N-carbamoyl lower alkyl-N,N-di-lower alkylammonino; lower cycloalkyl; lower alkylcarbonyl wherein the alkyl portion of lower alkylcarbonyl is optionally substituted by a halogen atom, cyano, hydroxyl, carbamoyl, amino, formylamino, lower alkylcarbonylamino, aminosulfonylamino, lower alkylthio, lower alkoxy, lower cycloalkyl, N,N-di-lower alkylamino, or N-carbamoyl lower alkyl-N,N-di-lower alkylammonino; carbamoyl; aryl optionally substituted by amino optionally substituted by one or two lower alkyl groups; lower alkylthio wherein the alkyl portion of lower alkylthio is optionally substituted by amino, hydroxyl, azide, a halogen atom, cyano, carbamoyl, formylamino, lower alkylcarbonylamino, aminosulfonylamino, or lower alkylthio; morpholinyl; lower alkylsulfonyl; or formyl; n is an integer of 0 to 4; and Hy represents a four- to seven-membered monocyclic or nine- or ten-membered bicyclic saturated or unsaturated heterocyclic group having one to four hetero-atoms selected from nitrogen, oxygen, and sulfur atoms, the saturated or unsaturated heterocyclic group represented by Hy is optionally substituted by a halogen atom; cyano; lower alkyl wherein one or more hydrogen atoms on the lower alkyl group are optionally substituted by groups selected from a halogen atom; hydroxyl; carbamoyl; carboxylmethyl-substituted carbamoyl; amino; N,N-di-lower alkylamino; aryl optionally substituted by amino; a monocyclic or bicyclic heterocyclic group containing one or more hetero-atoms selected from nitrogen, oxygen, and sulfur atoms, optionally substituted by aminosulfonyl or carboxyl;

carboxyl; imino; lower alkoxycarbonyl; lower alkylcarbonyl; aminosulfonylamino; amino lower alkylthio; lower alkylsulfonyl; (N,N-di-lower alkylamino)sulfonylamino; N′-(N,N-di-lower alkylamino)sulfonyl-N′-lower alkylamino; halogenated lower alkylcarbonyl; N-aminosulfonylpiperidinyl; and cyano; lower alkylthio wherein one or more hydrogen atoms on the alkyl group are optionally substituted by a group selected from a halogen atom, hydroxyl, carbamoyl, amino, and aryl; lower alkylsulfonyl wherein one or more hydrogen atoms on the alkyl group are optionally substituted by a group selected from a halogen atom, hydroxyl, carbamoyl, amino, 1-iminoethylamino, and aryl; hydroxyl; lower alkoxy; hydroxyaminophenyl-substituted lower alkoxy; halogenated lower alkoxy; aminophenyl-substituted lower alkoxy; formyl; lower alkylcarbonyl; arylcarbonyl; carboxyl; lower alkoxycarbonyl; carbamoyl; N-lower alkylcarbamoyl; N,N-di-lower alkylaminocarbonyl; amino; N-lower alkylamino; N,N-di-lower alkylamino; formylamino; lower alkylcarbonylamino; aminosulfonylamino; (N-lower alkylamino)sulfonylamino-; (N,N-di-lower alkylamino)sulfonylamino; aryl; or a monocyclic or bicyclic heterocyclic group containing one or more hetero-atoms selected from nitrogen, oxygen, and sulfur atoms, optionally substituted by aminosulfonyl or carboxyl, or a pharmaceutically acceptable salt, ester or pro-drug thereof.

A beta-lactam compound or composition of the invention can be a beta-lactam corresponding to Beta-Lactam II:

Beta-Lactam II

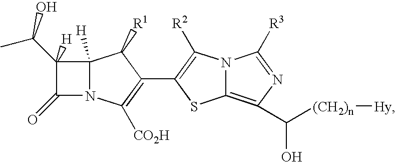

wherein R¹ represents a hydrogen atom or methyl, R² and R³, which may be the same or different, each represent a hydrogen atom; a halogen atom; lower alkyl optionally substituted by a halogen atom, cyano, hydroxyl, carbamoyl, amino, formylamino, lower alkylcarbonylamino, aminosulfonylamino, lower alkylthio, lower alkoxy, lower cycloalkyl, N,N-di-lower alkylamino, or N-carbamoyl lower alkyl-N,N-di-lower alkylammonino; lower cycloalkyl; lower alkylcarbonyl wherein the alkyl portion of lower alkylcarbonyl is optionally substituted by a halogen atom, cyano, hydroxyl, carbamoyl, amino, formylamino, lower alkylcarbonylamino, aminosulfonylamino, lower alkylthio, lower alkoxy, lower cycloalkyl, N,N-di-lower alkylamino, or N-carbamoyl lower alkyl-N,N-di-lower alkylammonino; carbamoyl; aryl optionally substituted by amino optionally substituted by one or two lower alkyl groups; lower alkylthio wherein the alkyl portion of lower alkylthio is optionally substituted by amino, hydroxyl, azide, a halogen atom, cyano, carbamoyl, formylamino, lower alkylcarbonylamino, aminosulfonylamino, or lower alkylthio; morpholinyl; lower alkylsulfonyl; or formyl; n is an integer of 0 to 4, and Hy represents a four- to seven-membered monocyclic or nine- or ten-membered bicyclic saturated or unsaturated heterocyclic group having one to four hetero-atoms selected from nitrogen, oxygen, and sulfur atoms, the saturated or unsaturated heterocyclic group represented by Hy is optionally substituted by a halogen atom; cyano; lower alkyl wherein one or more hydrogen atoms on the lower alkyl group are optionally substituted by groups selected from a halogen atom; hydroxyl; carbamoyl; carboxylmethyl-substituted carbamoyl; amino; N,N-di-lower alkylamino; aryl optionally substituted by amino; a monocyclic or bicyclic heterocyclic group containing one or more hetero-atoms selected from nitrogen, oxygen, and sulfur atoms, optionally substituted by aminosulfonyl or carboxyl; carboxyl; imino; lower alkoxycarbonyl; lower alkylcarbonyl; aminosulfonylamino; amino lower alkylthio; lower alkylsulfonyl; (N,N-di-lower alkylaamino)sulfonylamino; N′-(N,N-di-lower alkylamino)sulfonyl-N′-lower alkylamino; halogenated lower alkylcarbonyl; N-aminosulfonylpiperidinyl; and cyano; lower alkylthio wherein one or more hydrogen atoms on the alkyl group are optionally substituted by a group selected from a halogen atom, hydroxyl, carbamoyl, amino, and aryl; lower alkylsulfonyl wherein one or more hydrogen atoms on the alkyl group are optionally substituted by a group selected from a halogen atom, hydroxyl, carbamoyl, amino, 1-iminoethylamino, and aryl; hydroxyl; lower alkoxy; hydroxyaminophenyl-substituted lower alkoxy; halogenated lower alkoxy; aminophenyl-substituted lower alkoxy; formyl; lower alkylcarbonyl; arylcarbonyl; carboxyl; lower alkoxycarbonyl; carbamoyl; N-lower alkylcarbamoyl; N,N-di-lower alkylaminocarbonyl; amino; N-lower alkylamino; N,N-di-lower alkylaamino; formylamino; lower alkylcarbonylamino; aminosulfonylamino; (N-lower alkylamino)sulfonylamino-; (N,N-di-lower alkylamino)sulfonylamino; aryl; or a monocyclic or bicyclic heterocyclic group containing one or more hetero-atoms selected from nitrogen, oxygen, and sulfur atoms, optionally substituted by aminosulfonyl or carboxyl, or a pharmaceutically acceptable salt, ester, or pro-drug thereof.

In one example, the beta-lactam compound has the following structure: 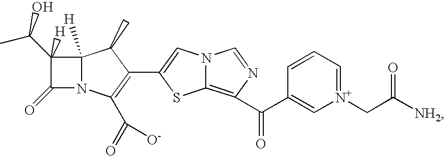
or is a pharmaceutically acceptable salt, ester, or prodrug thereof.

In one example, the compound useful in the invention is, or the composition useful in the invention includes an aminomethylcycline compound. For example, the aminomethylcycline compound can be 7-methylamino-9-(2,2-dimethyl-propyl)aminomethylcycline or a pharmaceutically acceptable salt, ester, or prodrug thereof.

The antimicrobial compound can be, for example, dalbavancin or a pharmaceutically acceptable salt, ester, or prodrug thereof.

The antimicrobial compound can be, for example, daptomycin or a pharmaceutically acceptable salt, ester, or prodrug thereof.

The antimicrobial compound can be, for example, garenoxacin or a pharmaceutically acceptable salt, ester, or prodrug thereof.

The antimicrobial compound can be, for example, gatifloxacin or a pharmaceutically acceptable salt, ester, or prodrug thereof.

The antimicrobial compound can be, for example, gemifloxacin or a pharmaceutically acceptable salt, ester, or prodrug thereof.

The antimicrobial compound can be, for example, levofloxacin or a pharmaceutically acceptable salt, ester, or prodrug thereof.

The antimicrobial compound can be, for example, moxifloxacin or a pharmaceutically acceptable salt, ester, or prodrug thereof.

The antimicrobial compound can be, for example, oritavancin or a pharmaceutically acceptable salt, ester, or prodrug thereof.

The antimicrobial compound can be, for example, televancin or a pharmaceutically acceptable salt, ester, or prodrug thereof.

The antimicrobial compound can be, for example, an oxazolidinone.

For example, the oxazolidinone compound can be linezolid or a pharmaceutically acceptable salt, ester, or prodrug thereof.

For example, the oxazolidinone compound can be one of the following compounds:

| | |
|---|---|
| A | 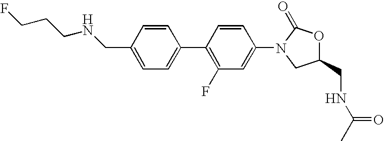 |
| | (5S)-N-(3-{2-Fluoro-4′-[(3-fluoro-propylamino)-methyl]-biphenyl-4- |
| | yl}-2-oxo-oxazolidin-5-ylmethyl)-acetamide |
| B | 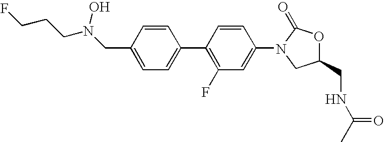 |
| | (5S)-N-[3-(2-Fluoro-4′-{[(3-fluoro-propyl)-hydroxy-amino]-methyl}- |
| | biphenyl-4-yl)-2-oxo-oxazolidin-5-ylmethyl]-acetamide |
| C | 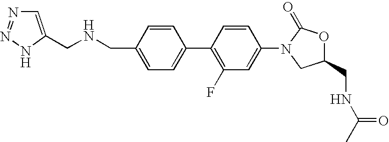 |
| | N-[3-(2-Fluoro-4′-{[(3H-[1,2,3]triazol-4-ylmethyl)-amino]-methyl}- |
| | biphenyl-4-yl)-2-oxo-oxazolidin-5-(S)-ylmethyl]-acetamide |
| D | 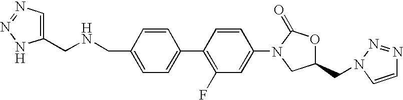 |
| | 3-(2-Fluoro-4′-{[(3H-[1,2,3]triazol-4-ylmethyl)-amino]-methyl}- |
| | biphenyl-4-yl)-5-(R)-[1,2,3]triazol-1-ylmethyl-oxazolidin-2-one |

or a pharmaceutically acceptable salt, ester, or prodrug thereof.

The antimicrobial compound can be, for example 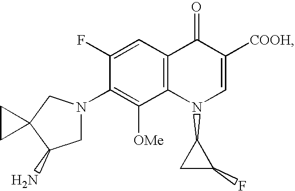
or a salt, ester, or prodrug thereof.

The antimicrobial compound can be, for example

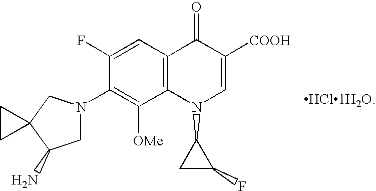

For example, the compound can be a crystalline compound of 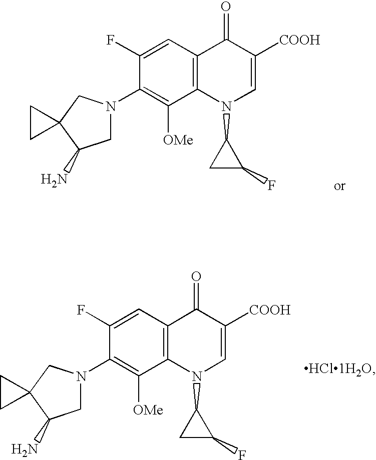
wherein the compound exhibits characteristic peaks in the vicinity of angles of diffraction (2θ) of 6.9, 10.5, 14.4, 23.1, 26.9, and 27.8(°) when subjected to powder X-ray diffractometry.

The invention relates to a composition comprising an antimicrobial agent described herein, for use as described herein, wherein the composition includes from about 0.1 to about 1500 mg. of the antimicrobial agent.

For example, a composition of the invention includes about 25 mg, or about 50 mg, or about 75 mg, or about 100 mg, or about 125 mg, or about 150 mg, or about 175 mg, or about 200 mg, or about 225 mg, or about 250 mg, or about 275 mg, or about 300 mg, or about 325, or about 350 mg, or about 375 mg, or about 400 mg, or about 425 mg, or about 450 mg, or about 475 mg, or about 500 mg, or about 525 mg, or about 550 mg, or about 575 mg, or about 600 mg, or about 625 mg, or about 650 mg, or about 675 mg, or about 700 mg, or about 725 mg, or about 750 mg, or about 775 mg, or about 800 mg, or about 825 mg, or about 850 mg, or about 875 mg, or about 900 mg, or about 925 mg, or about 950 mg, or about 975 mg, or about 1000 mg, or about 1025 mg, or about 1050, mg, or about 1075 mg, or about 1100 mg, or about 1125 mg, or about 1150 mg, or about 1175 mg, or about 1200 mg, or about 1225 mg, or about 1250 mg, or about 1275 mg, or about 1300 mg, or about 1325 mg, or about 1350 mg, or about 1375 mg, or about 1400 mg, or about 1425 mg, or about 1450 mg, or about 1475 mg, or about 1500 mg of the antimicrobial agent as described herein.

In general, the compounds useful in the methods of the present invention are administered, or administration is commenced, from about 24 hours prior to up to immediately before the surgical or invasive medical procedure. In other embodiments, the compounds useful in the methods of the present invention are administered, or administration is commenced, from about 6 hours prior to up to immediately before the surgical or invasive medical procedure. In other embodiments, the compounds useful in the methods of the present invention are administered, or administration is commenced, from about 4 hours prior to up to immediately before the surgical or invasive medical procedure. In other embodiments, the compounds useful in the methods of the present invention are administered, or administration is commenced, from about 2 hours prior to up to immediately before the surgical or invasive medical procedure. In other embodiments, the compounds useful in the methods of the present invention are administered, or administration is commenced, from about 1 hour prior to up to immediately before the surgical or invasive medical procedure. In some embodiments, the compounds useful in the methods of the present invention are administered, or administration is commenced, from about 30 minutes prior to up to immediately before the surgical or invasive medical procedure.

The foregoing and other aspects and embodiments of the present invention can be more fully understood by reference to the following detailed description and claims.

### BRIEF DESCRIPTION OF THE FIGURES

FIG. 1 shows a powder X-ray diffraction pattern of crystalline D-glucitol 1-(6-amino-3,5-difluoro-2-pyridinyl)-8-chloro-6-fluoro-1,4-hydro-7-(3-hydroxy-1-azetidinyl)-4-oxo-3-quinolinecarboxylate (salt).

FIG. 2 shows a powder X-ray diffraction pattern of D-glucitol 1-(6-amino-3,5-difluoro-2-pyridinyl)-8-chloro-6-fluoro-1,4-dihydro-7-(3-hydroxy-1-azetidinyl)-4-oxo-3-quinolinecarboxylate trihydrate (salt).

### DETAILED DESCRIPTION OF THE INVENTION

The present invention provides methods for reducing the risk of infection due to surgical procedures or other invasive medical procedures. The present invention further provides methods for preventing infection due to surgical procedures or other invasive medical procedures. The present invention further provides methods of peri-operative prophylaxis.

1. Definitions

The term “patient”, as used herein, means the human or animal (in the case of an animal, more typically a mammal) subject that would be subjected to a surgical or invasive medical procedure. Such patient or subject could be considered to be in need of the methods of reducing the risk of or preventing the infection due to a surgical procedure or an invasive medical procedure. Such patient or subject can also be considered to be in need of peri-operative prophylaxis.

The term “preventing”, as used herein means, to completely or almost completely stop an infection from occurring due to a surgical or invasive medical procedure, for example when the patient or subject is predisposed to an infection or at risk of contracting an infection. Preventing can also include inhibiting, i.e. arresting the development, of an infection, and relieving or ameliorating, i.e. causing regression of the infection, for example when an infection may already be present prior to surgery or an invasive medical procedure, e.g., a nonlimiting example of which would be when the patient or subject has been injured and has a dirty or contaminated wound.

The term “reducing the risk of”, as used herein, means to lower the likelihood or probability of an infection occurring due to a surgical or invasive medical procedure, for example when the patient or subject is predisposed to an infection or at risk of contracting an infection.

The terms “surgical procedure” or “invasive medical procedure”, as used herein mean any surgical procedure, as understood by one of ordinary skill in the art or other medical procedure that involves an entry or contact with the patient or subject, or that potentially compromises the integrity of the skin or other tissues. Nonlimiting examples of surgical procedures include: cardiothoracic surgery, vascular surgery, colon surgery, hip or knee arthroplasty, biliary surgery, vaginal or abdominal hysterectomy, percutaneous gastronomy, repair of long bone fractures, dental surgery, oral surgery, ear, nose or throat surgery, Cesarean section, etc. Nonlimiting examples of invasive medical procedures include: non-surgical dental procedures (e.g., tooth repairs and cleanings), bladder catheter insertions, insertion of nasal cannulas, biopsies, dermatological procedures, hair transplants, removal of a foreign object from the skin, eye, nasal, ear, or other body cavities, etc. It should be understood that the distinction between surgical procedures and invasive medical procedures can be somewhat arbitrary and is meant to provide a general guideline for defining procedures where a patient or subject could be at risk of contracting an infection from the procedure.

The methods of the present invention, whereby one prevents or reduces the risk of an infection due to a surgical or invasive medical procedure, can also be referred to by the term “peri-operative prophylaxis” or the acronym, “POP”.

As used herein, the term “effective amount” refers to an amount of a compound, or a combination of compounds, of the present invention effective when administered alone or in combination as an antimicrobial agent. For example, an effective amount refers to an amount of the compound present in a composition, a formulation or on a medical device given to a recipient patient or subject sufficient to elicit biological activity, for example, anti-infective activity, such as e.g., anti-microbial activity, anti-bacterial activity, anti-fungal activity, anti-viral activity, or anti-parasitic activity.

A combination of compounds optionally is a synergistic combination. Synergy, as described, for example, by Chou and Talalay, Adv. Enzyme Regul. vol. 22, pp. 27-55 (1984), occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased anti-proliferative and/or anti-infective effect, or some other beneficial effect of the combination compared with the individual components.

The term “prophylactically effective amount” means an effective amount of a compound or compounds, of the present invention that is administered to prevent or reduce the risk of an infection due to a surgical procedure or an invasive medical procedure.

With respect to the chemical compounds useful in the present invention, the following terms can be applicable:

The term “substituted,” as used herein, means that any one or more hydrogens on the designated atom is replaced with a selection from the indicated group, provided that the designated atom's normal valency is not exceeded, and that the substitution results in a stable compound. When the substituent is keto (i.e., ═O), then 2 hydrogens on the atom are replaced. Ring double bonds, as used herein, are double bonds that are formed between two adjacent ring atoms (e.g., C═C, C═N, or N═N).

With respect to any chemical compounds, the present invention is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The chemical compounds described herein can have asymmetric centers. Compounds of the present invention containing an asymmetrically substituted atom can be isolated in optically active or racemic forms. If is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and can be isolated as a mixture of isomers or as separated isomeric forms. All chiral, diastereomeric, racemic, and geometric isomeric forms of a structure are intended, unless the specific stereochemistry or isomeric form is specifically indicated. All processes used to prepare compounds of the present invention and intermediates made therein are, where appropriate, considered to be part of the present invention. All tautomers of shown or described compounds are also, where appropriate, considered to be part of the present invention.

When a bond to a substituent is shown to cross a bond connecting two atoms in a ring, then such substituent can be bonded to any atom in the ring. When a substituent is listed without indicating the atom via which such substituent is bonded to the rest of the compound of a given formula, then such substituent can be bonded via any atom in such substituent. Combinations of substituents and/or variables are permissible, but only if such combinations result in stable compounds.

When an atom or a chemical moiety is followed by a subscripted numeric range (e.g., C₁₋₆), the invention is meant to encompass each number within the range as well as all intermediate ranges. For example, “C₁₋₆ alkyl” is meant to include alkyl groups with 1, 2, 3, 4, 5, 6, 1-6, 1-5, 1-4, 1-3, 1-2, 2-6, 2-5, 2-4, 2-3, 3-6, 3-5, 3-4, 4-6, 4-5, and 5-6 carbons.

As used herein, “alkyl” is intended to include both branched and straight-chain saturated aliphatic hydrocarbon groups having the specified number of carbon atoms. For example, C₁₋₆ alkyl is intended to include C₁, C₂, C₃, C₄, C₅, and C₆ alkyl groups. Examples of alkyl include, but are not limited to, methyl, ethyl, n-propyl, i-propyl, n-butyl, s-butyl, t-butyl, n-pentyl, s-pentyl, and n-hexyl.

As used herein, “alkenyl” is intended to include hydrocarbon chains of either straight or branched configuration having one or more carbon-carbon double bonds occurring at any stable point along the chain. For example, C₂₋₆ alkenyl is intended to include C₂, C₃, C₄, C₅, and C₆ alkenyl groups. Examples of alkenyl include, but are not limited to, ethenyl and propenyl.

As used herein, “alkynyl” is intended to include hydrocarbon chains of either straight or branched configuration having one or more carbon-carbon triple bonds occurring at any stable point along the chain. For example, C₂₋₆ alkynyl is intended to include C₂, C₃, C₄, C₅, and C₆ alkynyl groups. Examples of alkynyl include, but are not limited to, ethynyl and propynyl.

Furthermore, “alkyl”, “alkenyl”, and “alkynyl” are intended to include moieties which are diradicals, i.e., having two points of attachment. A nonlimiting example of such an alkyl moiety that is a diradical is —CH₂CH₂—, i.e., a C₂ alkyl group that is covalently bonded via each terminal carbon atom to the remainder of the molecule.

As used herein, “halo” or “halogen” refers to fluoro, chloro, bromo, and iodo. “Counterion” is used to represent a small, negatively charged species such as fluoride, chloride, bromide, iodide, hydroxide, acetate, and sulfate.

As used herein, “carbocycle” or “carbocyclic ring” is intended to mean any stable monocyclic, bicyclic, tricyclic, or higher order cyclic ring having the specified number of carbons, any of which can be saturated, unsaturated, or aromatic, recognizing that rings with certain numbers of members cannot be bicyclic or tricyclic, e.g., a 3-membered ring can only be a monocyclic ring. For example, a C₃₋₁₄ carbocycle is intended to mean a monocyclic, bicyclic, tricyclic, or higher order cyclic ring having 3, 4, 5, 6, 7, 8, 9, 10, 11, 12, 13, or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, cyclooctyl, cyclooctenyl, cyclooctadienyl, adamantyl, fluorenyl, phenyl, naphthyl, indanyl, anthryl, phenanthryl, and tetrahydronaphthyl. Bridged rings are also included in the definition of carbocycle, including, for example, [3.3.0]bicyclooctane, [4.3.0]bicyclononane, [4.4.0]bicyclodecane, and [2.2.2]bicyclooctane. A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring can also be present on the bridge. Fused (e.g., naphthyl and tetrahydronaphthyl) and spiro rings are also included.

It should be understood that included in the definition of “carbocycle” and “carbocyclic ring” are “aromatic carbocycles” and “aromatic carbocyclic rings,” which are “aryl” groups. In the case of bicyclic aromatic carbocyclic rings, only one of the rings needs to be aromatic (e.g., tetrahydronaphthyl), though both can be (e.g., naphthyl). Similarly, in the case of tricyclic or higher order aromatic carbocyclic rings, only one of the rings needs to be aromatic, although tricyclic or higher order aromatic carbocycles having more than one aromatic ring are included (e.g., fluorenyl). Examples of aromatic carbocycles include, but are not limited to, phenyl, naphthyl, tetrahydronaphthyl, indanyl, indenyl, phenanthryl, anthryl, fluorenyl, pentalenyl, azulyl, chrysyl, pyryl, tetracyl, fluranthyl, coronyl, and hexahelicyl.

As used herein, the term “heterocycle” or “heterocyclic” is intended to mean any stable monocyclic, bicyclic, tricyclic, or higher order cyclic ring (recognizing that rings with certain numbers of members cannot be bicyclic or tricyclic, e.g., a 3-membered ring can only be a monocyclic ring), which is saturated, unsaturated, or aromatic and comprises carbon atoms and one or more ring heteroatoms, e.g., 1 or 1-2 or 1-3 or 1-4 or 1-5 or 1-6 heteroatoms, independently selected from nitrogen, oxygen, and sulfur. A bicyclic or tricyclic heterocycle can have one or more heteroatoms located in one ring, or the heteroatoms can be located in more than one ring. The nitrogen and sulfur heteroatoms can optionally be oxidized (i.e., N—O, S(O), and S(O)₂. When a nitrogen atom is included in the ring it is either N or NH, depending on whether or not it is attached to a double bond in the ring (i.e., a hydrogen is present if needed to maintain the tri-valency of the nitrogen atom). The nitrogen atom can be substituted or unsubstituted (e.g., N or NR wherein R is H or another substituent, as defined). The heterocyclic ring can be attached to its pendant group at any heteroatom or carbon atom that results in a stable structure. The heterocyclic rings can be substituted on carbon or on a nitrogen atom if the resulting compound is stable. A nitrogen in the heterocycle can optionally be quatemized. It is preferred that when the total number of S and O atoms in the heterocycle exceeds 1, then these heteroatoms are not adjacent to one another. Bridged rings are also included in the definition of heterocycle. A bridged ring occurs when one or more atoms (i.e., C, O, N, or S) link two non-adjacent carbon or nitrogen atoms. Bridges include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring can also be present on the bridge. Spiro and fused rings are also included.

As used herein, the term “aromatic heterocycle” or “heteroaryl” is intended to mean a stable monocyclic, bicyclic, or higher order aromatic heterocyclic ring, which consists of carbon atoms and one or more heteroatoms, e.g., 1 or 1-2 or 1-3 or 1-4 or 1-5 or 1-6 heteroatoms, independently selected from nitrogen, oxygen, and sulfur. For example, an aromatic heterocycle or heteroaryl can be a 5, 6, 7, 8, 9, 10, 11, or 12-membered monocyclic or bicyclic aromatic heterocyclic ring, recognizing that rings with certain numbers of members cannot be a bicyclic aromatic, e.g., a 5-membered ring can only be a monocyclic aromatic ring. In the case of bicyclic heterocyclic aromatic rings, only one of the two rings needs to be aromatic (e.g., 2,3-dihydroindole), though both can be (e.g., quinoline). The second ring can also be fused or bridged as defined above for heterocycles. The nitrogen atom can be substituted or unsubstituted (i.e., N or NR wherein R is H or another substituent, as defined). The nitrogen and sulfur heteroatoms can optionally be oxidized (i.e., N→O, S(O), and S(O)₂).

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, D-glucitol (methylgluconate), edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

The pharmaceutically acceptable salts of the present invention can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed. (Mack Publishing Company, 1990). For example, salts can include, but are not limited to, the hydrochloride and acetate salts of the aliphatic amine-containing, hydroxyl amine-containing, and imine-containing compounds of the present invention.

Additionally, the compounds of the present invention, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

The compounds of the present invention can also be prepared as esters, for example pharmaceutically acceptable esters. For example a carboxylic acid function group in a compound can be converted to its corresponding ester, e.g., a methyl, ethyl, or other ester. Also, an alcohol group in a compound can be converted to its corresponding ester, e.g., an acetate, propionate, or other ester.

The compounds of the present invention can also be prepared as prodrugs, for example pharmaceutically acceptable prodrugs. Since prodrugs are known to enhance numerous desirable qualities of pharmaceuticals (e.g., solubility, bioavailability, manufacturing, etc.) the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, or sulflhydryl group is bonded to any group that, when the prodrug of the present invention is administered to a mammalian subject, cleaves to form a free hydroxyl, free amino, or free sulfhydryl group, respectively. Examples of prodrugs include, but are not limited to, acetate, formate, and benzoate derivatives of alcohol and amine functional groups in the compounds of the present invention.

“Stable compound” and “stable structure” are meant to indicate a compound that is sufficiently robust to survive isolation, and as appropriate, purification from a reaction mixture, and formulation into an efficacious therapeutic agent.

In the specification, the singular forms also include the plural, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

2. Methods of the Invention

The present invention provides a method of reducing the risk of a microbial infection in a patient or subject having a surgical or invasive medical procedure, said method comprising administering a prophylactically effective amount of an antimicrobial compound to said patient or subject prior to said surgical or invasive medical procedure.

The present invention also provides a method of preventing a microbial infection in a patient or subject having a surgical or invasive medical procedure, said method comprising administering a prophylactically effective amount of an antimicrobial compound to said patient or subject prior to said surgical or invasive medical procedure.

The present invention also provides a method of peri-operative prophylaxis in a patient in need thereof, said method comprising administering a prophylactically effective amount of an antimicrobial compound to said patient or subject prior to a surgical or invasive medical procedure.

As discussed above, microbial infections at the site of surgery or other invasive medical procedures are a potentially serious risk for patients. It is been found that compounds of the present invention are useful for reducing the risk of or preventing these microbial infections, or for providing peri-operative prophylaxis.

The methods of the present invention can be usefully applied to patients, whether human or animal, undergoing a wide range of surgical or invasive medical procedures. Nonlimiting examples of surgical procedures include: cardiothoracic surgery, vascular surgery, colon surgery, hip or knee arthroplasty, biliary surgery, vaginal or abdominal hysterectomy, percutaneous gastronomy, repair of long bone fractures, dental surgery, oral surgery, ear, nose or throat surgery, Cesarean section, etc. Nonlimiting examples of invasive medical procedures include: non-surgical dental procedures (e.g., tooth repairs and cleanings), bladder catheter insertions, insertion of nasal cannulas, biopsies, dermatological procedures, hair transplants, removal of a foreign object from the skin, eye, nasal, ear, or other body cavities, etc.

Additionally, it is found that the methods of the present invention are useful for reducing the risk of or preventing infections, nonlimiting example of said infections including skin infections, abdominal infections, urinary tract infections, bacteremia, septicemia, endocarditis, infections associated with an atrio-ventricular shunt, vascular access infections, meningitis, peritoneal infections, bone infections, deep sternal wound infections, joint infections, infection associated with a catherization, infections associated with placement of a stent, infections associated with placement of a prosthetic device, Gram-negative bacterial infections, Sraphylococcus aureus infections, including those caused by methicillin-resistant Staphylococcus aureus bacteria, enterococcal infections, including those caused by vancomycin-resistant enterococci, Bacteriodes infections, other aneaerobic bacterial organism caused infections, and linezolid-resistant infections.

In practicing the methods of the present invention, it is desired that the blood and or tissue level in the patient or subject, of the compound used to provide the effect be of an appropriate level for a sufficient time interval for the surgical or invasive medical procedure to be completed - in other words for the effective dosage of the compound to be present for an appropriate period before, during, and after the surgical or invasive medical procedure. Also, because it often takes a finite amount of time to achieve such blood or tissue levels, it is important that the compound is administered at some appropriate time prior to the patient or subject undergoing the surgical or invasive medical procedure. The appropriate time for administration of the compound will depend upon the pharmacokinetic profile of the compound and its formulation, route of administration, time required for completing administration, patient characteristics, type of surgery or invasive medical procedure, desired clinical outcome, etc. In general, the compounds useful in the methods of the present invention are administered, or administration is commenced, from about 24 hours prior to up to immediately before the surgical or invasive medical procedure. In other embodiments, the compounds useful in the methods of the present invention are administered, or administration is commenced, from about 6 hours prior to up to immediately before the surgical or invasive medical procedure. In other embodiments, the compounds useful in the methods of the present invention are administered, or administration is commenced, from about 4 hours prior to up to immediately before the surgical or invasive medical procedure. In other embodiments, the compounds useful in the methods of the present invention are administered, or administration is commenced, from about 2 hours prior to up to immediately before the surgical or invasive medical procedure. In other embodiments, the compounds useful in the methods of the present invention are administered, or administration is commenced, from about 1 hour prior to up to immediately before the surgical or invasive medical procedure. In some embodiments, the compounds useful in the methods of the present invention are administered, or administration is commenced, from about 30 minutes prior to up to immediately before the surgical or invasive medical procedure.

Furthermore, in certain instances, it may be necessary or desirable to provide an additional dosage of the compound prior to, during, or after the surgical or invasive medical procedure. Examples where an additional dosage may be required can include where the commencement of the surgical or invasive medical procedure is delayed relative to the initial administration of the compound, or where the surgical or invasive medical procedure takes longer to complete than originally anticipated, or where it is found from monitoring of blood or tissues or other appropriate analyses of the patient or subject that further administration of compound is warranted. Furthermnore, in certain instances, it may be necessary or desirable to continuously administer the compound during some portion or all of the surgical or invasive medical procedure, and yet in further instances it may be necessary or desirable to continue to administer the compound for some time period after completion of the surgical or invasive medical procedure.

The compounds used in the methods of the present invention can be administered by any of the common modes of administration, including, e.g., intravenous administration, oral administration, subcutaneous administration, parenteral administration, intramuscular administration, opthalmic administration, nasal administration or by inhalation, otic administration, vaginal administration, rectal administration, etc. Many of the patients or subjects requiring administration of the compounds are sedated or anesthetized in preparation for the surgery or invasive medical procedure or will later be seated or anesthetized. Many of the patients or subjects may be unable to swallow. Also, oral or other forms of administration may be contraindicated because of the surgery or invasive medical procedure. In these foregoing instances intravenous administration is generally preferred. Furthermore, intravenous administration provides the healthcare provider with more control over the time and rate of administration of the compound.

3. Compounds of the invention

A wide range of antimicrobial compounds can be used in the methods, compositions, and uses of the present invention. These antimicrobial compounds can provide their therapeutic effect by a variety of biochemical or biophysical mechanisms. Compounds useful in the present invention can include those which bind to or modulate ribosomal RNA, for example bacterial ribosomal RNA. Compounds also useful in the present invention can include those which bind to or modulate the large ribosomal subunit, for example the large ribosomal subunit of a bacterial organism. Compounds also useful in the present invention can include those which bind to or modulate DNA topoisomerases, for example bacterial DNA topoisomerases. Compounds also useful in the present invention can include those which bind to or modulate bacterial DNA gyrase, for example bacterial DNA gyrase, i.e. gyrase being an example of a topoisomerase. Compounds also useful in the present invention can include those which bind to or modulate bacterial topoisomerase IV.

Useful antimicrobial agents include antibacterial agents, antifungal agents, anti-viral agents, and anti-parasitic agents. Useful chemical classes of compounds include those selected from macrolides, ketolides, streptogramin As, streptogramin Bs, chloramphenicol and chloramphenicol derivatives, fluorfenicol and fluorfenicol derivatives, glycopeptides, pleuromutilins, aminoglycosides, beta-lactams and carbapenems (including carbapenems with a 7-acylated imidazo[5-1,b]thiazole-2-yl group directly attached to the carbapenem moiety of the C-2 position), cephalosporins, lincosamides, quinolones, fluoroquinolones, pyridonecarboxylic acid derivatives, benzoheterocyclic compounds, aminomethylcycline compounds, dalbavancin, daptomycin, garenoxacin, gatifloxacin, gemifloxacin, levofloxacin, moxifloxacin, oritavancin, oxazolidinones (e.g., linezolid and other oxazolidinones), televancin, and mixtures thereof. It should be noted that compounds useful herein can in some instances be classified in more than one way. The description or classification of a compound or compounds is not intended to limit that compound or compounds, but is being done for the sake of convenience.

The compounds useful in the present invention can include the pharmaceutically acceptable salts, esters, or prodrugs thereof. The invention further provides methods for synthesizing any one of the compounds of the present invention. The invention also provides pharmaceutical compositions comprising an effective amount of one or more of the compounds of the present invention and a pharmaceutically acceptable carrier. The present invention further provides methods for making these pharmaceutical compositions.

Pyridonecarboxylic Acid Derivatives

Pyridonecarboxylic acid derivatives useful herein are described, including their synthesis, formulation, and use, in U.S. Pat. No. 6,156,903, to Yazaki et al., issued Dec. 5, 2000 and its certificate of correction of Dec. 11, 2001; U.S. Pat. No. 6,133, 284, to Yazaki et al., issued Oct. 17, 2000; U.S. Pat. No. 5,998, 436, to Yazaki et al., issued Dec. 7, 1999 and its certificate of corrections of Jan. 23, 2001 and Dec. 17, 2002; PCT Application No. WO 2006/042034, to Abbott Laboratories, published Apr. 20, 2006, PCT Application No. WO 2006/015194, to Abbott Laboratories, published Feb. 9, 2006; PCT Application No. WO 01/34595, to Wakunaga Pharmaceutical Co., Ltd., published May 17, 2001; and PCT Application No. WO 97/11068, to Wakunaga Pharmaceutical Co., Ltd., published Mar. 27, 1997.

Pyridonecarboxylic acid derivatives of the methods, compositions, and uses of the present invention include compounds corresponding to the following structure (Pyridonecarboxylic Acid Derivative 1)
Pyridonecarboxylic Acid Derivative 1 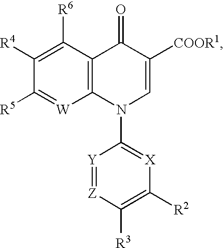
wherein R¹ represents a hydrogen atom or a carboxyl protective group; R² represents a hydroxyl group, a lower alkoxy group, or a substituted or unsubstituted amino group; R³ represents a hydrogen atom or a halogen atom; R⁴ represents a hydrogen atom or a halogen atom; R⁵ represents a halogen atom or an optionally substituted saturated cyclic amino group; R⁶ represents a hydrogen atom, a halogen atom, a nitro group, or an optionally protected amino group; X, Y and Z may be the same or different and respectively represent a nitrogen atom, CH or CR⁷ (wherein R⁷ represents a lower alkyl group, a halogen atom, or a cyano group), with the proviso that at least one of X, Y and Z represent a nitrogen atom, and W represents a nitrogen atom or CR⁸ (wherein R⁸ represents a hydrogen atom, a halogen atom, or a lower alkyl group), and with the proviso that when R¹ represents a hydrogen atom, R² represents an amino group, R³ and R⁴ represent a fluorine atom, R⁶ represents a hydrogen atom, X represents a nitrogen atom, Y represents CR⁷ (wherein R⁷ represents a fluorine atom), Z represents CH, and W is CR⁸ (wherein R⁸ represents a chlorine atom), then R⁵ is not a 3-hydroxyazetidine-1-yl group; or a pharmaceutically acceptable salt, ester, or prodrug thereof.

As described in the foregoing paragraph, when R¹ is a carboxyl protective group, it may be any carboxylate ester residue which cleaves relatively easily to generate the corresponding free carboxyl group. Exemplary carboxyl protective groups include those which may be eliminated by hydrolysis, catalytic reduction, and other treatments under mild conditions such as lower alkyl groups such as methyl group, ethyl group, n-propyl group, i-propyl group, n-butyl group, i-butyl group, t-butyl group, pentyl group, hexyl group, and heptyl group; lower alkenyl groups such as vinyl group, allyl group, 1-propenyl group, butenyl group, pentenyl group, hexenyl group, and heptenyl group; aralkyl groups such as benzyl group; and aryl groups such as phenyl group and naphthyl group; and those which may be readily eliminated in the body such as lower alkanoyloxy lower alkyl groups such as acetoxymethyl group and pivaloyloxymethyl group; lower alkoxycarbonyloxy lower alkyl group such as methoxycarbonyloxymethyl group and 1-ethoxycarbonyloxyethyl group; lower alkoxymethyl group such as methoxymethyl group; lactonyl group such as phthalidyl; di-lower alkylamino lower alkyl group such as 1-dimethylaminoethyl group; and (5-methyl-2-oxo-1,3-dioxole-4-yl)methyl group.

It is noted that the substituents R¹, R², R³, R^{4,} R^{5,} R¹, R¹, R¹, R^{9,} A, J , J2, J3, W, X, Y, Z, e, f; and g are defined herein for convenience with respect to the chemical structure for the pyridonecarboxylic acid derivatives, e.g., Pyridonecarboxylic Acid Derivative 1, and do not refer to other substituents for other compounds of the present invention.

In other embodiments, the present invention relates to a method, composition, or use for a pyridonecarboxylic acid derivative of structure Pyridonecarboxylic Acid Derivative 1, wherein W is CR⁸, wherein R⁸ represents a hydrogen atom, a halogen atom, or a lower alkyl group.

In other embodiments, the present invention relates to a method, composition, or use for a pyridonecarboxylic acid derivative of structure Pyridonecarboxylic Acid Derivative 1, wherein R⁵ is a group represented by the following formula (a) or (b): 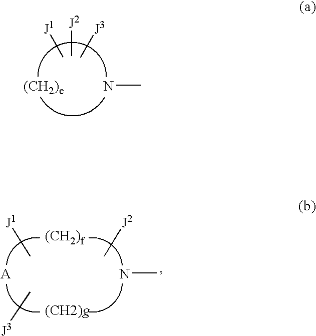
wherein A represents an oxygen atom, sulfur atom or NR⁹ (wherein R⁹ represents hydrogen atom or a lower alkyl group), e represents a number from 3 to 5, f represents a number from I to 3, g represents a number from 0 to 2, J¹, J² and J³, which may be the same or different from one another, represent a hydrogen atom, hydroxyl group, lower alkyl group, amino lower alkyl group, amino group, lower alkylamino group, lower alkoxy group, or a halogen atom.

In other embodiments, the present invention relates to a method, composition, or use for a pyridonecarboxylic acid derivative of structure Pyridonecarboxylic Acid Derivative 1, wherein R⁵ is a group represented by formula (a).

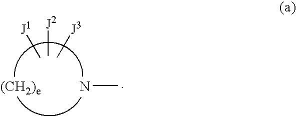

In other embodiments, the present invention relates to a method, composition, or use for a pyridonecarboxylic acid derivative of structure Pyridonecarboxylic Acid Derivative 1, wherein e in the formula (a) is 3 or 4.

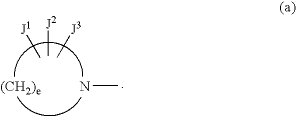

In other embodiments, the present invention relates to a method, composition, or use for a pyridonecarboxylic acid derivative of structure Pyridonecarboxylic Acid Derivative 1, wherein R¹ is a hydrogen atom; R² is an amino group, lower alkylamino group, or a di-lower alkylaamino group; R³ is a halogen atom; R⁴ is a halogen atom; R⁶ is hydrogen atom; X is a nitrogen atom; Y and Z are CH or CR⁷ (wherein R⁷ is a lower alkyl group or a halogen atom); and W is CR⁸ (wherein R⁸ is a halogen atom or a lower alkyl group).

In other embodiments, the present invention relates to a method, composition, or use for a pyridonecarboxylic acid derivative of structure Pyridonecarboxylic Acid Derivative 1, wherein R² is amino group; R³ is fluorine atom; R⁴ is a fluorine atom; Y is CF; Z is CH; W is CR⁸ (wherein R⁸is a chlorine atom, bromine atom or a methyl group), and e in formula (a) is 3.

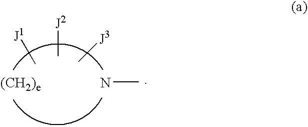

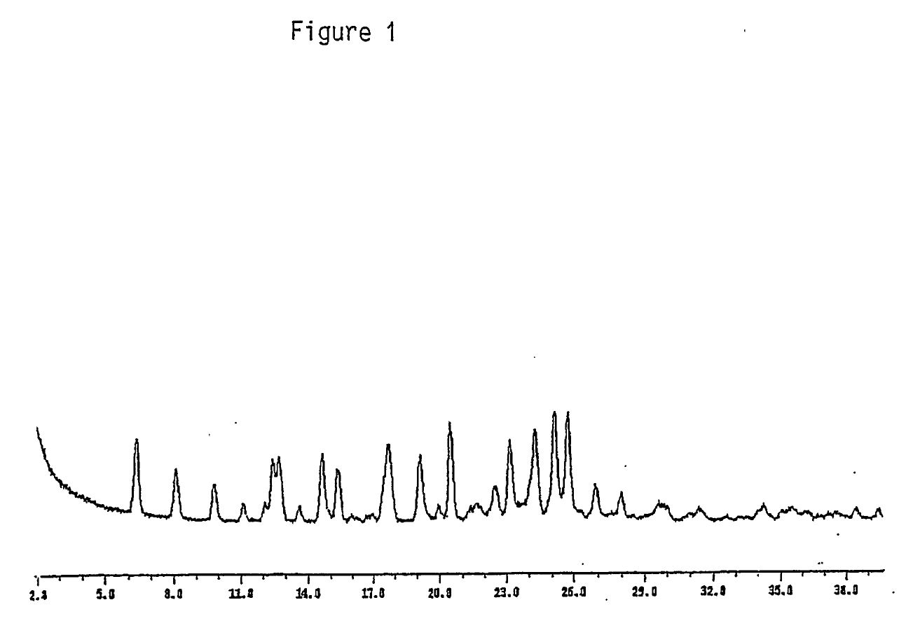

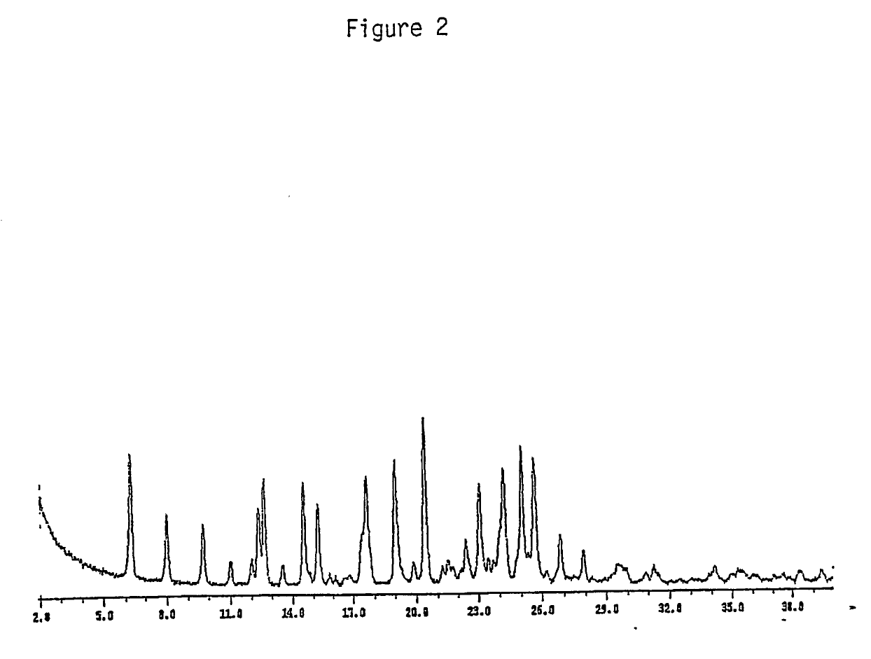

In other embodiments, the present invention relates to a method, composition, or use wherein said pyridonecarboxylic acid corresponds to the following structure: 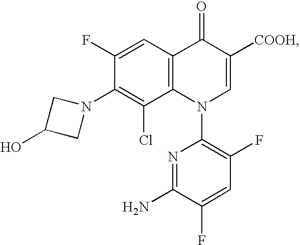
or a pharmaceutically acceptable salt, ester, or prodrug thereof. This foregoing pyridonecarboxylic acid is also known by the publicly disclosed code names ABT-492 and WQ 3034 and also by the chemical name 1-(6-amino-3,5-difluoro-2-pyridinyl)-8-chloro-6-fluoro-1,4-dihydro-7-(3-hydroxy-1-azetidinyl)-4-oxo-3-quinolinecarboxylic acid or 1-(6-amino-3,5-difluoro-2-pyridinyl)-8-chloro-6-fluoro-1,4-dihydro-7-(3-hydroxyazetidin-1-yl)-4-oxo-3-quinolinecarboxylic acid. Furthermore, WO 2006/042034, cited above discloses the D-glucitol salt of this compound [D-glucitol 1-(6-amino-3,5-difluoro-2-pyridinyl)-8-chloro-6-fluoro-1,4-dihydro-7-(3-hydroxy-1-azetidinyl)-4-oxo-3-quinolinecarboxylate (salt)]and the trihydrate of the D-glucitol salt of this compound [D-glucitol 1-(6-amino-3,5-difluoro-2-pyridinyl)-8-chloro-6-fluoro-1,4-dihydro-7-(3-hydroxy-1-azetidinyl)-4-oxo-3-quinolinecarboxylate trihydrate (salt)]. WO 2006/042034 also discloses a crystalline form of the D-glucitol salt characterized when measured at about 25° C. with Cu-Ka radiation, by the powder diffraction pattern shown in FIG. 1 and a crystalline form of the D-glucitol salt trihydrate when measured at about 25° C. with Cu-Ka radiation, by the powder diffraction pattern shown in FIG. 2. These D-glucitol salts are useful in the present invention.

Benzoheterocyclic Compounds

Benzoheterocyclic compounds useful herein are described, including their synthesis, formulation, and use, in U.S. Pat. No. 6,753,333 B2, to De Souza et al., issued Jun. 22, 2004; U.S. Pat. No. 6,750,224 B1, to Patel et al, issued Jun. 15, 2004 and its certificate of correction of Nov. 2, 2004; U.S. Pat. No. 6,664,267 B1, to de Souza et al., issued Dec. 16, 2003; U.S. Pat. No. 6,608,078 B2, to De Souza et al., issued Aug. 19, 2003; U.S. Pat. No. 6,514,986 B2 to De Souza et al., issued Feb. 4, 2003; U.S. Pat. No. 4,552,879 to Ishikawa et al., issued Nov. 12, 1985; and U.S. Pat. No. 4,399,134 to Ishikawa et al., issued Aug. 16, 1983.

Benzoheterocyclic compounds of the methods, compositions, and uses of the present invention include compounds corresponding to the following structure (Benzoheterocyclic Compound I)
Benzoheterocyclic Compound I 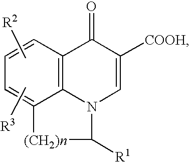
wherein R¹ represents a hydrogen atom or a lower alkyl group; R² represents a hydrogen atom or a halogen atom; R³ represents a 1-pyrrolidinyl group which may be substituted with a hydroxymethyl group, a 1,2,5,6-tetrahydro-1-pyridyl group, or a group of the formula 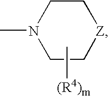
where R⁴ represents a hydrogen atom, a lower alkyl group, a lower alkoxy group, a hydroxy group, a phenyl-lower alkyl group, a lower alkanoyloxy group, an amino group which may be substituted with a lower alkyl group or a lower alkanoyl group, an oxo group, or a carbamoyl group; Z represents an oxygen atom, a sulfur atom or a methylene group; and m is 1 or 2; and n is an integer of 1 or 2; or a pharmaceutically acceptable salt ester or prodrug thereof.

It is noted that the substituents R¹, R², R³, R⁴, Z, m, and n are defined herein for convenience with respect to the chemical structure for the benzoheterocyclic compounds, e.g., benzoheterocyclic compound (I) and do not refer to other substituents for other compounds of the present invention.

In other embodiments, the present invention relates to a method, composition, or use for a benzoheterocyclic of structure Benzoheterocyclic Compound I, wherein n is 2.

In other embodiments, the present invention relates to a method, composition, or use for a benzoheterocyclic of structure Benzoheterocyclic Compound I, wherein n is 1.

In other embodiments, the present invention relates to a method, composition, or use for a benzoheterocyclic of structure Benzoheterocyclic Compound I, wherein R³ represents a group of the formula 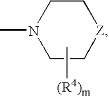
where R⁴ represents a hydrogen atom, a lower alkyl group, a lower alkoxy group, a hydroxy group, a phenyl-lower alkyl group, a lower alkanoyloxy group, an amino group which may be substituted with a lower alkyl group or a lower alkanoyl group, an oxo group, or a carbamoyl group; Z represents an oxygen atom, a sulfur atom or a methylene group; and m is 1 or 2; and n is 1.

In other embodiments, the present invention relates to a method, composition, or use for a benzoheterocyclic of structure Benzoheterocyclic Compound I, wherein R³ represents a 1-pyrrolidinyl group which may be substituted with a hydroxymethyl group or a 1,2,5,6-tetrahydro-1-pyridyl group.

In other embodiments, the present invention relates to a method, composition, or use for a benzoheterocyclic of structure Benzoheterocyclic Compound I, wherein R⁴ represents a hydrogen atom, a hydroxy group or a lower alkanoyloxy group and the position at which the group of the formula 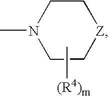
where R⁴ represents a hydrogen atom, a lower alkyl group, a lower alkoxy group, a hydroxy group, a phenyl-lower alkyl group, a lower alkanoyloxy group, an amino group which may be substituted with a lower alkyl group or a lower alkanoyl group, an oxo group, or a carbamoyl group; Z represents an oxygen atom, a sulfur atom or a methylene group; and m is 1 or 2; and n is 1, is attached is the 8-position.

In other embodiments, the present invention relates to a method, composition, or use for a benzoheterocyclic of structure Benzoheterocyclic Compound I, wherein R⁴ represents a lower alkyl group, a lower alkoxy group, a phenyl-lower alkyl group, an amino group which may be substituted with a lower alkyl group or a lower alkanoyl group, an oxo group, a carbamoyl group, and the position at which the group of the formula 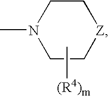
where R⁴ represents a hydrogen atom, a lower alkyl group, a lower alkoxy group, a hydroxy group, a phenyl-lower alkyl group, a lower alkanoyloxy group, an amino group which may be substituted with a lower alkyl group or a lower alkanoyl group, an oxo group, or a carbamoyl group; Z represents an oxygen atom, a sulfur atom or a methylene group; and m is 1 or 2; and n is 1, is attached is the 8-position.

In other embodiments, the present invention relates to a method, composition, or use for a benzoheterocyclic of structure Benzoheterocyclic Compound I, wherein R² represents a halogen atom.

In other embodiments, the present invention relates to a method, composition, or use for a benzoheterocyclic of structure Benzoheterocyclic Compound I, wherein R² represents a hydrogen atom.

In other embodiments, the present invention relates to a method, composition, or use for a benzoheterocyclic of structure Benzoheterocyclic Compound I, wherein R² represents a fluorine atom and the position at which the fluorine atom is attached is the 9-position.

In other embodiments, the present invention relates to a method, composition, or use for a benzoheterocyclic of structure Benzoheterocyclic Compound I, wherein R² represents a chlorine atom and the position at which the fluorine atom is attached is the 9-position.

In other embodiments, the present invention relates to a method, composition, or use for a benzoheterocyclic of structure Benzoheterocyclic Compound I, wherein R¹ represents a lower alkyl group.

In other embodiments, the present invention relates to a method, composition, or use for a benzoheterocyclic of structure Benzoheterocyclic Compound I, wherein R¹ represents a methyl group.

In other embodiments, the present invention relates to a method, composition, or use for a benzoheterocyclic of structure Benzoheterocyclic Compound I, wherein R² represents a fluorine atom attached to the 9-position and R¹ represents a methyl group.

In other embodiments, the present invention relates to a method, composition, or use for a benzoheterocyclic of structure Benzoheterocyclic Compound I, wherein R¹ represents a methyl group, R² represents a fluorine atom attached to the 9-position and the position at which the group represented by R³ is attached is the 8-position.

In other embodiments, the present invention relates to a method, composition, or use for a benzoheterocyclic of structure Benzoheterocyclic Compound I, wherein the position at which R³ is attached is the 9-position.

In other embodiments, the present invention relates to a method, composition, or use for a benzoheterocyclic of structure Benzoheterocyclic Compound I, wherein R¹ represents a methyl group, R² represents a fluorine atom attached to the 8-position.

In other embodiments, the present invention relates to a method, composition, or use for a benzoheterocyclic of structure Benzoheterocyclic Compound I, wherein R¹ represents a methyl group, R² represents a chlorine atom attached to the 8-position.

In other embodiments, the present invention relates to a method, composition, or use wherein said benzoheterocyclic compound is 9-fluoro-8-(4-hydroxy-l -piperidyl)-5-methyl-6,7-dihydro-1- oxo-1H,5H-benzo[i,j]quinolizine-2-carboxylic acid or a pharmaceutically acceptable salt, ester, or prodrug thereof.

In other embodiments, the present invention relates to a method, composition, or use wherein said benzoheterocyclic compound is S-(−)-9-fluoro-6,7-dihydro-8-(4-hydroxypiperidin-1-yl)-5-methyl-1-oxo-1H,5H-benzo[i,j]quinolizine-2-carboxylic acid or a pharmaceutically acceptable salt, ester, or prodrug thereof. The foregoing compound is also known by the chemical name nadifloxacin.

In other embodiments, the present invention relates to a method, composition, or use wherein said benzoheterocyclic compound is S-(−)-9-fluoro-6,7-dihydro-8-(4-hydroxypiperidin-1-yl)-5-methyl-1-oxo-1H,5H-benzo[i,j]quinolizine-2-carboxylic acid arginine salt.

In other embodiments, the present invention relates to a method, composition, or use wherein said benzoheterocyclic compound is a specific polymorph or crystalline form of S-(−)-9-fluoro-6,7-dihydro-8-(4-hydroxypiperidin-1-yl)-5-methyl-1-oxo-1 H,5H-benzo[i,j]quinolizine-2-carboxylic acid arginine salt.

In other embodiments, the present invention relates to a method, composition, or use wherein said benzoheterocyclic compound is S-(−)-9-fluoro-6,7-dihydro-8-(4-hydroxypiperidin-1-yl)-5-methyl-1-oxo-1H,5H-benzo[i,j]quinolizine-2-carboxylic acid arginine salt having the following X-ray diffraction data: (2θ): 10.16, 11.78, 12,52, 16.00, 18.94, 19.66, 20.36, 21.28, 21.92, 22.52, 24.74, 25.28, 30.74.

In other embodiments, the present invention relates to a method, composition, or use wherein said benzoheterocyclic compound is S-(−)-9-fluoro-6,7-dihydro-8-(4-hydroxypiperidin-1-yl)-5-methyl-1-oxo-1H,5H-benzo[i,j]quinolizine-2-carboxylic acid arginine salt having the following X-ray diffraction data: (2θ): 18.28, 18.8, 19.8, 20.12, 20.62, 21.10, 21.44, 21.88, 22.6, 23.02.

In other embodiments, the present invention relates to a method, composition, or use wherein said benzoheterocyclic compound is S-(−)-9-fluoro-6,7-dihydro-8-(4-hydroxypiperidin-1-yl)-5-methyl-1-oxo-1H,5H-benzo[i,j]quinolizine-2-carboxylic acid arginine salt having the following X-ray diffraction data: (2θ): 14.02±0.2, 14.82±0.2, 19.28±0.2, 22.12±0.2, 22.96±0.2, 23.46±0.2, 28.36±0.2.

With respect to specific polymorph or crystalline forms of the benzoheterocyclic compounds, examples being the arginine salts, a publicly disclosed code name for such a compound is WCK 771.

Beta-Lactams

Beta-lactams, for example carbapenems, examples of which are carbapenems with a 7-acylated imidazo[5-1,b]thiazole-2-yl group directly attached to the carbapenem moiety of the C-2 position, useful herein are described, including their synthesis, formulation, and use, in M. Kurazano et al., “In Vitro Activities of ME1036 (CP5609), a Novel Parenteral Carbapenem, Against Methicillin-Resistant Staphylococci”, Antimicrobial Agents and Chemotherapy, vol. 48, no. 8, pp. 2831-2837 (August 2004); U.S. Patent Application Publication No. US 2004/0038967 A1, to Kano et al., published Feb. 26, 2004; PCT Application No. WO 2004/055027, to Meiji Seika Kaisha, Ltd., published July 1, 2004; and PCT Application No. WO 02/042312, to Meiji Seika Kaisha, Ltd., published May 30, 2002.

Beta-lactam compounds of the methods, compositions, and uses of the present invention include compounds corresponding to the following structure (Beta-Lactam I)
Beta-Lactam I 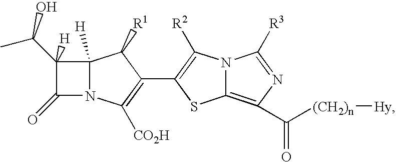
wherein R¹ represents a hydrogen atom or methyl, R² and R³, which may be the same or different, each represent a hydrogen atom; a halogen atom; lower alkyl optionally substituted by a halogen atom, cyano, hydroxyl, carbamoyl, amino, formylamino, lower alkylcarbonylamino, aminosulfonylamino, lower alkylthio, lower alkoxy, lower cycloalkyl, N,N-di-lower alkylamino, or N-carbamoyl lower alkyl-N,N-di-lower alkylammonino; lower cycloalkyl; lower alkylcarbonyl wherein the alkyl portion of lower alkylcarbonyl is optionally substituted by a halogen atom, cyano, hydroxyl, carbamoyl, amino, formylamino, lower alkylcarbonylamino, aminosulfonylamino, lower alkylthio, lower alkoxy, lower cycloalkyl, N,N-di-lower alkylamino, or N-carbamoyl lower alkyl-N,N-di-lower alkylammonino; carbamoyl; aryl optionally substituted by amino optionally substituted by one or two lower alkyl groups; lower alkylthio wherein the alkyl portion of lower alkylthio is optionally substituted by amino, hydroxyl, azide, a halogen atom, cyano, carbamoyl, formylamino, lower alkylcarbonylamino, aminosulfonylamino, or lower alkylthio; morpholinyl; lower alkylsulfonyl; or formyl; n is an integer of 0 to 4, and Hy represents a four- to seven-membered monocyclic or nine- or ten-membered bicyclic saturated or unsaturated heterocyclic group having one to four hetero-atoms selected from nitrogen, oxygen, and sulfur atoms, the saturated or unsaturated heterocyclic group represented by Hy is optionally substituted by a halogen atom; cyano; lower alkyl wherein one or more hydrogen atoms on the lower alkyl group are optionally substituted by groups selected from a halogen atom; hydroxyl; carbamoyl; carboxylmethyl-substituted carbamoyl; amino; N,N-di-lower alkylamino; aryl optionally substituted by amino; a monocyclic or bicyclic heterocyclic group containing one or more hetero-atoms selected from nitrogen, oxygen, and sulfur atoms, optionally substituted by aminosulfonyl or carboxyl; carboxyl; imino; lower alkoxycarbonyl; lower alkylcarbonyl; aminosulfonylamino; amino lower alkylthio; lower alkylsulfonyl; (N,N-di-lower alkylamino)sulfonylamino; N′-(N,N-di-lower alkylamino)sulfonyl-N′-lower alkylamino; halogenated lower alkylcarbonyl; N-aminosulfonylpiperidinyl; and cyano; lower alkylthio wherein one or more hydrogen atoms on the alkyl group are optionally substituted by a group selected from a halogen atom, hydroxyl, carbamoyl, amino, and aryl; lower alkylsulfonyl wherein one or more hydrogen atoms on the alkyl group are optionally substituted by a group selected from a halogen atom, hydroxyl, carbamoyl, amino, 1-iminoethylamino, and aryl; hydroxyl; lower alkoxy; hydroxyaminophenyl-substituted lower alkoxy; halogenated lower alkoxy; aminophenyl-substituted lower alkoxy; formyl; lower alkylcarbonyl; arylcarbonyl; carboxyl; lower alkoxycarbonyl; carbamoyl; N-lower alkylcarbamoyl; N,N-di-lower alkylaminocarbonyl; amino; N-lower alkylamino; N,N-di-lower alkylamino; formylamino; lower alkylcarbonylamino; aminosulfonylamino; (N-lower alkylamino)sulfonylamino-; (N,N-di-lower alkylamino)sulfonylamino; aryl; or a monocyclic or bicyclic heterocyclic group containing one or more hetero-atoms selected from nitrogen, oxygen, and sulfur atoms, optionally substituted by aminosulfonyl or carboxyl, or a pharmaceutically acceptable salt, ester or pro-drug thereof.

It is noted that the substituents R¹, R², R³, Hy, and n are defined herein for convenience with respect to the chemical structure for the beta-lactams or carbapenems, e.g., Beta-Lactam I and Beta-Lactam II, and do not refer to other substituents for other compounds of the present invention.

In other embodiments, the present invention relates to a method, composition, or use for a beta-lactam of structure Beta-Lactam I, wherein R¹ represents a hydrogen atom or methyl, R² and R³, which may be the same or different, each represent a hydrogen atom; a halogen atom; lower alkyl optionally substituted by a halogen atom, cyano, hydroxyl, carbamoyl, amino, formylamino, lower alkylcarbonylamino, aminosulfonylamino, or lower alkylthio; lower alkylcarbonyl wherein the alkyl portion of lower alkylcarbonyl is optionally substituted by a halogen atom, cyano, hydroxyl, carbamoyl, amino, formylamino, lower alkylcarbonylamino, aminosulfonylamino, or lower alkylthio; carbamoyl; aryl; or lower alkylthio wherein the alkyl portion of lower alkylthio is optionally substituted by a halogen atom, cyano, hydroxyl, carbamoyl, amino, formylamino, lower alkylcarbonylamino, aminosulfonylamino, or lower alkylthio, n is an integer of 0 to 4, and Hy represents a four- to seven-membered monocyclic or nine- or ten-membered bicyclic saturated or unsaturated heterocyclic group containing one to four hetero-atoms selected from nitrogen, oxygen, and sulfur atoms, the saturated or unsaturated heterocyclic group represented by Hy is optionally substituted by a halogen atom; cyano; lower alkyl wherein one or more hydrogen atoms on the lower alkyl group are optionally substituted by groups selected from a halogen atom, hydroxyl, carbamoyl, amino, aryl, and a monocyclic or bicyclic heterocyclic group containing one or more hetero-atoms selected from nitrogen, oxygen, and sulfur atoms; lower alkylthio wherein one or more hydrogen atoms on the alkyl group are optionally substituted by groups selected from a halogen atom, hydroxyl, carbamoyl, amino, and aryl; lower alkylsulfonyl wherein one or more hydrogen atoms on the alkyl group are optionally substituted by groups selected from a halogen atom, hydroxyl, carbamoyl, amino, and aryl; hydroxyl; lower alkoxy; formyl; lower alkylcarbonyl; arylcarbonyl; carboxyl; lower alkoxycarbonyl; carbamoyl; N-lower alkylcarbamoyl; N,N-di-lower alkylaminocarbonyl; amino; N-lower alkylamino; N,N-di-lower alkylamino; formylamino; lower alkylcarbonylamino; aminosulfonylamino; (N-lower alkylamino)sulfonylamino; (N,N-di-lower alkylamino)sulfonylamino; aryl; or a monocyclic or bicyclic heterocyclic group containing one or more hetero-atoms selected from nitrogen, oxygen, and sulfur atoms.

In other embodiments, the present invention relates to a method, composition, or use of a beta-lactam of structure Beta-Lactam I wherein R¹ represents a hydrogen atom or methyl, R² and R³, which may be the same or different, each represent a hydrogen atom, a halogen atom, optionally substituted lower alkyl, lower cycloalkyl, lower alkylcarbonyl, carbamoyl, optionally substituted aryl, optionally substituted lower alkylthio, morpholinyl, lower alkylsulfonyl, or formyl, n is an integer of 0 to 2, and Hy represents a group selected from optionally substituted pyridinyl, optionally substituted pyridinium-yl, optionally substituted tetrahydropyridinyl, optionally substituted thiazolyl, optionally substituted pyrimidinyl, optionally substituted thienyl, optionally substituted quinolinyl, optionally substituted quinolinium-yl, optionally substituted isoquinolinyl, optionally substituted dihydroisoquinolinyl, optionally substituted piperazinyl, optionally substituted piperidinyl, optionally substituted indolyl, optionally substituted thiomorpholinyl, optionally substituted imidazolyl, and optionally substituted pyrrolidinyl.

In other embodiments, the present invention relates to a method, composition, or use of a beta-lactam of structure Beta-Lactam I wherein R¹ represents a hydrogen atom or methyl, R² and R³, which may be the same or different, each represent a hydrogen atom, a halogen atom, optionally substituted lower alkyl, optionally substituted lower alkylcarbonyl, carbamoyl, aryl, or optionally substituted lower alkylthio, n is an integer of 0 to 4, and Hy represents a group selected from optionally substituted pyridinyl, optionally substituted pyridinium-yl, optionally substituted tetrahydropyridinyl, optionally substituted thiazolyl, optionally substituted pyrimidinyl, optionally substituted thienyl, optionally substituted quinolinyl, optionally substituted quinolinium-yl, and optionally substituted pyrrolidinyl.

In other embodiments, the present invention relates to Beta-lactam compounds of the methods, compositions, and uses of the present invention include compounds corresponding to the following structure (Beta-Lactam II)
Beta-Lactam II 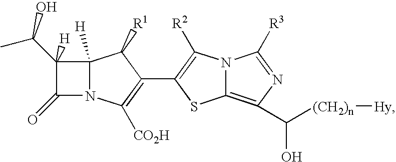
wherein R¹ represents a hydrogen atom or methyl, R² and R³, which may be the same or different, each represent a hydrogen atom; a halogen atom; lower alkyl optionally substituted by a halogen atom, cyano, hydroxyl, carbamoyl, amino, formylamino, lower alkylcarbonylamino, aminosulfonylamino, lower alkylthio, lower alkoxy, lower cycloalkyl, N,N-di-lower alkylamino, or N-carbamoyl lower alkyl-N,N-di-lower alkylammonino; lower cycloalkyl; lower alkylcarbonyl wherein the alkyl portion of lower alkylcarbonyl is optionally substituted by a halogen atom, cyano, hydroxyl, carbamoyl, amino, formylamino, lower alkylcarbonylamino, aminosulfonylamino, lower alkylthio, lower alkoxy, lower cycloalkyl, N,N-di-lower alkylamino, or N-carbamoyl lower alkyl-N,N-di-lower alkylammonino; carbamoyl; aryl optionally substituted by amino optionally substituted by one or two lower alkyl groups; lower alkylthio wherein the alkyl portion of lower alkylthio is optionally substituted by amino, hydroxyl, azide, a halogen atom, cyano, carbamoyl, formylamino, lower alkylcarbonylamino, aminosulfonylamino, or lower alkylthio; morpholinyl; lower alkylsulfonyl; or formyl; n is an integer of 0 to 4, and Hy represents a four- to seven-membered monocyclic or nine- or ten-membered bicyclic saturated or unsaturated heterocyclic group having one to four hetero-atoms selected from nitrogen, oxygen, and sulfur atoms, the saturated or unsaturated heterocyclic group represented by Hy is optionally substituted by a halogen atom; cyano; lower alkyl wherein one or more hydrogen atoms on the lower alkyl group are optionally substituted by groups selected from a halogen atom; hydroxyl; carbamoyl; carboxylmethyl-substituted carbamoyl; amino; N,N-di-lower alkylamino; aryl optionally substituted by amino; a monocyclic or bicyclic heterocyclic group containing one or more hetero-atoms selected from nitrogen, oxygen, and sulfur atoms, optionally substituted by aminosulfonyl or carboxyl; carboxyl; imino; lower alkoxycarbonyl; lower alkylcarbonyl; aminosulfonylamino; amino lower alkylthio; lower alkylsulfonyl; (N,N-di-lower alkylamino)sulfonylamino; N′-(N,N-di-lower alkylamino)sulfonyl-N′-lower alkylamino; halogenated lower alkylcarbonyl; N-aminosulfonylpiperidinyl; and cyano; lower alkylthio wherein one or more hydrogen atoms on the alkyl group are optionally substituted by a group selected from a halogen atom, hydroxyl, carbamoyl, amino, and aryl; lower alkylsulfonyl wherein one or more hydrogen atoms on the alkyl group are optionally substituted by a group selected from a halogen atom, hydroxyl, carbamoyl, amino, 1-iminoethylamino, and aryl; hydroxyl; lower alkoxy; hydroxyaminophenyl-substituted lower alkoxy; halogenated lower alkoxy; aminophenyl-substituted lower alkoxy; formyl; lower alkylcarbonyl; arylcarbonyl; carboxyl; lower alkoxycarbonyl; carbamoyl; N-lower alkylcarbamoyl; N,N-di-lower alkylaminocarbonyl; amino; N-lower alkylamino; N,N-di-lower alkylamino; formylamino; lower alkylcarbonylamino; aminosulfonylamino; (N-lower alkylamino)sulfonylamino-; (N,N-di-lower alkylamino)sulfonylamino; aryl; or a monocyclic or bicyclic heterocyclic group containing one or more hetero-atoms selected from nitrogen, oxygen, and sulfur atoms, optionally substituted by aminosulfonyl or carboxyl, or a pharmaceutically acceptable salt, ester, or pro-drug thereof.

In other embodiments, the present invention relates to a method, composition, or use of a beta-lactam of structure Beta-Lactam II, wherein R¹ represents a hydrogen atom or methyl, R² and R³, which may be the same or different, each represent a hydrogen atom, a halogen atom, lower alkyl optionally substituted by a halogen atom, cyano, hydroxyl, carbamoyl, amino, formylamino, lower alkylcarbonylamino, aminosulfonylamino, or lower alkylthio; lower alkylcarbonyl wherein the alkyl portion of lower alkylcarbonyl is optionally substituted by a halogen atom, cyano, hydroxyl, carbamoyl, amino, formylamino, lower alkylcarbonylaamino, aminosulfonylamino, or lower alkylthio; carbamoyl; aryl; or lower alkylthio wherein the alkyl portion of lower alkylthio is optionally substituted by a halogen atom, cyano, hydroxyl, carbamoyl, amino, formylamino, lower alkylcarbonylamino, aminosulfonylamino, or lower alkylthio, n is an integer of 0 to 4, and Hy represents a four- to seven-membered monocyclic or nine- or ten-membered bicyclic saturated or unsaturated heterocyclic group containing one to four hetero-atoms selected from nitrogen, oxygen, and sulfur atoms, the saturated or unsaturated heterocyclic group represented by Hy is optionally substituted by a halogen atom; cyano; lower alkyl wherein one or more hydrogen atoms on the lower alkyl group are optionally substituted by groups selected from a halogen atom, hydroxyl, carbamoyl, amino, aryl, and a monocyclic or bicyclic heterocyclic group containing one or more hetero-atoms selected from nitrogen, oxygen, and sulfur atoms; lower alkylthio wherein one or more hydrogen atoms on the alkyl group are optionally substituted by groups selected from a halogen atom, hydroxyl, carbamoyl, amino, and aryl; lower alkylsulfonyl wherein one or more hydrogen atoms on the alkyl group are optionally substituted by groups selected from a halogen atom, hydroxyl, carbamoyl, amino, and aryl; hydroxyl; lower alkoxy; formyl; lower alkylcarbonyl; arylcarbonyl; carboxyl; lower alkoxycarbonyl; carbamoyl; N-lower alkylcarbamoyl; N,N-di-lower alkylaminocarbonyl; amino; N-lower alkylamino; N,N-di-lower alkylamino; formylamino; lower alkylcarbonylamino; aminosulfonylamino; (N-lower alkylamino)sulfonylamino; (N,N-di-lower alkylamino)sulfonylamino; aryl; or a monocyclic or bicyclic heterocyclic group containing one or more hetero-atoms selected from nitrogen, oxygen, and sulfur atoms.

In other embodiments, the present invention relates to a method, composition, or use of a beta-lactam of structure Beta-Lactam I or Beta-Lactam II, wherein the substituent on the lower alkyl and lower alkylcarbonyl groups optionally represented by R² and R³ is hydroxyl, lower alkoxy, N,N-di-lower alkylaamino, or N-carbamoyl lower alkyl-N,N-di-lower alkylammonino, the substituent on the aryl group optionally represented by R² and R³ is N,N-di-lower alkylamino, the substituent on the lower alkylthio group optionally represented by R² and R³ is amino, hydroxyl, or azide, and the substituent on the saturated or unsaturated heterocyclic ring represented by Hy is lower alkyl optionally substituted by carboxylmethyl-substituted carbamoyl, carbamoyl, phenyl, aminophenyl, N,N-di-lower alkylamino, amino, hydroxyl, morpholinyl, pyrrolidinyl, carboxyl, imino, amino lower alkylthio, lower alkoxycarbonyl, lower alkylcarbonyl, aminosulfonylamino, piperidinyl, lower alkylsulfonyl, (N,N-di-lower alkylamino)sulfonylamino, N′-(N,N-di-lower alkylamino)sulfonyl-N′-lower alkylamino, halogenated lower alkylcarbonyl, N-aminosulfonylpiperidinyl, or cyano; carbamoyl; pyridinyl; N-aminosulfonylpyrrolidinyl; 2-carboxypyrrolidinyl; phenyl; hydroxyl; lower alkoxy; hydroxyaminophenyl-substituted lower alkoxy; halogenated lower alkoxy; aminophenyl-substituted lower alkoxy; amino; carboxyl; lower alkylthio optionally substituted by amino; amino lower alkylthio; amino lower alkylsulfonyl; or 1-iminoethylamino lower alkylsulfonyl.

In other embodiments, the present invention relates to a method, composition, or use of a beta-lactam of structure Beta-Lactam I or Beta-Lactam II, wherein R¹ represents a hydrogen atom or methyl, R² and R³ represent a hydrogen atom, n is 0 (zero), and Hy represents pyridinium-yl having carbamoylmethyl at its 1-position.

In other embodiments, the present invention relates to a method, composition, or use of a beta-lactam of structure Beta-Lactam I or Beta-Lactam II, wherein n is 0 (zero).

In other embodiments, the present invention relates to a method, composition, or use of a beta-lactam of structure Beta-Lactam I or Beta-Lactam II, wherein R¹ represents methyl, and R² and R³ represent a hydrogen atom.

In other embodiments, the present invention relates to a method, composition, or use of a beta-lactam of structure Beta-Lactam I or Beta-Lactam II, wherein R¹ represents methyl, R² and R³ represent a hydrogen atom, n is 0 (zero), and Hy represents pyridinium-yl which optionally has carbamoyl lower alkyl, carboxyl lower alkyl, or aminosulfonylamino lower alkyl at its 1-position and amino lower alkylthio at other position than the 1-position.

In other embodiments, the present invention relates to a method, composition, or use of a beta-lactam of structure Beta-Lactam I or Beta-Lactam II, wherein R¹ represents methyl, R² and R³ represent a hydrogen atom, n is 0 (zero), and Hy represents pyridin-3-yl.

In other embodiments, the present invention relates to a method, composition, or use of a beta-lactam of structure Beta-Lactam I or Beta-Lactam II, wherein R¹ represents methyl, R² and R³ represent a hydrogen atom, n is 0 (zero), and Hy represents 1-carbamoylmethylpyridinium-3-yl.

In other embodiments, the present invention relates to a method, composition, or use of a beta-lactam of structure Beta-Lactam I or Beta-Lactam II, wherein R¹, R² and R³ represent a hydrogen atom, n is 0 (zero), and Hy represents I-carbamoylmethylpyridinium-3-yl.

In other embodiments, the present invention relates to a method, composition, or use of a beta-lactam of structure Beta-Lactam I or Beta-Lactam II, wherein R¹ represents methyl, R² and R³ represent a hydrogen atom, n is 0 (zero), and Hy represents 1-carbamoylmethyl-5-phenylpyridinium-3-yl.

In other embodiments, the present invention relates to a method, composition, or use of a beta-lactam of structure Beta-Lactam I or Beta-Lactam II, wherein R¹ represents methyl, R² and R³ represent a hydrogen atom, n is 0 (zero), and Hy represents (2S)-pyrrolidin-2-yl.

In other embodiments, the present invention relates to a method, composition, or use of a beta-lactam of structure Beta-Lactam I or Beta-Lactam II, wherein R¹ represents methyl, R² and R³ represent a hydrogen atom, n is 0 (zero), and Hy represents 1-carboxymethylpyridinium-3-yl.

In other embodiments, the present invention relates to a method, composition, or use of a beta-lactam of structure Beta-Lactam I or Beta-Lactam II, wherein R¹ represents methyl, R² and R³ represent a hydrogen atom, n is 0 (zero), and Hy represents 1-(2-aminosulfonylaminoethyl)pyridinium-3-yl.

In other embodiments, the present invention relates to a method, composition, or use wherein said beta-lactam or carbapenem corresponds to the following structure: 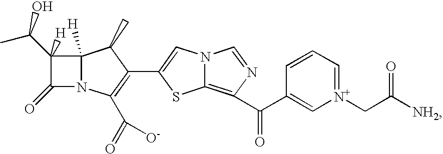
or a pharmaceutically acceptable salt, ester, or prodrug thereof. This foregoing beta-lactam or carbapenem is also known by the publicly disclosed code names ME1 036 and CP5609.
Aminomethylcycline Compounds

Aminomethylcycline compounds such as 7-methylamino-9-(2,2-dimethyl-propyl)aminomethylcycline and their pharmaceutically acceptable salts, esters, and prodrugs thereof, can be used in the methods, compositions, and uses of the present invention. The compound, 7-methylamino-9-(2,2-dimethyl-propyl)aminomethylcycline, is also known by the publicly disclosed code names PTK 0796 and BAY 73-6944. See U.S. Pat. No. 6,846,939 B2, to Nelson et al., issued Jan. 25, 2005; U.S. Patent Application No. US 2005/0070510 A1, to Draper et al., published Mar. 31, 2005; U.S. Patent Application No. US 2005/0026876 A1, to Nelson et al., published Feb. 3, 2005; U.S. Patent Application No. US 2005/0026875 A1, to Nelson et al., published Feb. 3, 2005; U.S. Patent Application No. US 2004/0242548 A1, to Draper et al., published Dec. 2, 2004; U.S. Patent Application No. US 2004/0214801 A1, to Nelson et al, published Oct. 28, 2004; U.S. Patent Application No. US 2004/0214800 A1, to Levy et al., published Oct. 28, 2004; U.S. Patent Application No. US 2004/0092490 A1, to Draper et al., published May 13, 2004; U.S. Patent Application No. US 2004/0063674 A1, to Levy et al., published Apr. 1, 2004; U.S. Patent Application No. US 2003/0166585 A1, to Draper et al., published Sep. 4, 2003; U.S. Patent Application No. US 2003/0125348 A1, to Nelson et al, published Jul. 3, 2003; PCT Application No. WO 2005/009944, to Paratek Pharmaceuticals, Inc., published Feb. 3, 2005; PCT Application No. WO 2004/091513, to Paratek Pharmaceuticals, Inc., published Oct. 28, 2004; PCT Application No. WO 2004/064728, to Paratek Pharmaceuticals, Inc., published Aug. 5, 2004; PCT Application No. WO 2004/038001, to Paratek Pharmaceuticals, Inc., published May 6, 2004; PCT Application No. WO 2004/038000, to Paratek Pharmaceuticals, Inc.; published May 6, 2004; PCT Application No. WO 03/075857, to Paratek Pharmaceuticals, Inc., published Sep. 18, 2003; PCT Application No. WO 03/005971, to Paratek Pharmaceuticals, Inc., published Jan. 23, 2003; PCT Application No. WO 02/072031, to Paratek Pharmaceuticals, Inc., published Sep. 19, 2002; and PCT Application No. WO 02/04406, to Trustees of Tufts College and Paratek Pharmaceuticals, Inc., published Jan. 17, 2002.

Dalbavancin

Dalbavancin and its pharmaceutically acceptable salts, esters, and prodrugs thereof, can be used in the methods, compositions, and uses of the present invention. Dalbavancin, which is a semisynthetic glycopeptide is also known by the publicly disclosed code names VER-001 and B1397. See G. Candiani et al., “In-Vitro and In-Vivo Antibacterial Activity of BI 397, a New Semi-Sinthetic Glycopeptide Antibiotic”, J. Antimicrob. Chemotherapy, 44, pp. 179-192 (1999); U.S. Patent Application No. US 2005/0090433 A1, to Colombo et al., published Apr. 28, 2005; U.S. Patent Application No. US 2005/0004050 A1, to Stogniew, published Jan. 6, 2005; U.S. Patent Application No. US 2004/0224908 A1, to Cavaleri et al., published Nov. 11, 2004; U.S. Patent Application No. US 2004/0220122 A1, to Cavaleri et al., published Nov. 4, 2004; U.S. Patent Application No. US 2004/0198715 A1, to Cavaleri et al., published Oct. 7, 2004.

Daptomycin

Daptomycin and its pharmaceutically acceptable salts, esters, and prodrugs thereof, can be used in the methods, compositions, and uses of the present invention. Daptomycin is sold under the tradename or proprietary Cubicin. See U.S. Pat. No. 6,852,689 B2, to Oleson, Jr. et al., issued Feb. 8, 2005; U.S. Pat. No. 6,468,967 B1, to Oleson, Jr. et al., issued Oct. 22, 2002; and U.S. Pat. No. 5,912,226, to Baker et al., issued Jun. 15, 1999; and PCT Application No. WO 00/18419, to Cubist Pharmaceuticals, Inc., published Apr. 6, 2000.

Garenoxacin

Garenoxacin and its pharmaceutically acceptable salts, esters, and prodrugs thereof, can be used in the methods, compositions, and uses of the present invention. Garenoxacin is also known as 1-cyclopropyl -8-(difluoromethoxy)-7-(1R)-(1-methyl-2,3-dihydro-1H-5-isoinodyl)-4-oxo-1,4-dihydro-3-uinolinecarboxylic acid methanesulfonate monohydrate and by the publicly disclosed code names T-3811 and BM 284756. See M. Takahata et al., “In Vitro and In Vivo Antimicrobial Activities of T-381 IME, a Novel Des-F(6)-Quinolone”, Antimicrobial Agents and Chemotherapy, vol. 43, no. 5, pp. 1077-1084 (1999); U.S. Pat. No. 6,025,370, to Todo et al, issued Feb. 15, 2000; and U.S. Pat. 5,935,952, to Todo et al., issued Aug. 10, 1999 and its certificate of correction of Dec. 5, 2000.

Gatifloxacin

Gatifloxacin and its pharmaceutically acceptable salts, esters, and prodrugs thereof, can be used in the methods, compositions, and uses of the present invention. Gatifloxacin is sold under the tradename or proprietary Tequin. See U.S. Pat. No. 6,589,955 B2, to Raghavan et al., issued Jul. 8, 2003; U.S. Pat. No. 5,880,283, to Matsumoto et al., issued Mar. 9, 1999; and U.S. Pat. No. 4,980,470, to Masuzawa et al., issued Dec. 25, 1990 and its certificate of correction of Aug. 11, 1992.

Gemifloxacin

Gemifloxacin and its pharmaceutically acceptable salts, esters, and prodrugs thereof, can be used in the methods, compositions, and uses of the present invention. Gemifloxacin is sold under the tradename or proprietary Factive. See U.S. Pat. No. 6,803,376 B1, to Appelbaum et al., issued Oct. 12, 2004; U.S. Pat. No. 6,723,734 B2, to Kim et al., issued Apr. 20, 2004; U.S. Patent No. 6,455,540 B1, to Citron et al., issued Sep. 24, 2002; U.S. Pat. No. 6,340,689 B1, to Dubois et al., issued Jan., 22, 2002 and its certificate of correction of Jun. 18, 2002; U.S. Pat. No. 6,331,550 B1, to Citron et al., issued Dec. 18, 2001; U.S. Pat. No. 6,262,071 B1, to Crabb et al., issued Jul. 17, 2001; U.S. Pat. No. 5,962,468, to Hong et al., issued Oct. 5, 1999 and its certificate of correction of May 9, 2000; U.S. Pat. No. 5,776,944, to Hong et al., issued Jul. 7, 1998; and U.S. Pat. No. 5,633,262, to Hong et al., issued May 27, 1997.

Levofloxacin

Levofloxacin and its pharmaceutically acceptable salts, esters, and prodrugs thereof, can be used in the methods, compositions, and uses of the present invention. Levofloxacin is sold under the tradename or proprietary Levaquin. See U.S. Pat. No. 5,053,407, to Hayakawa et al., issued Oct. 1, 1991 and its certificate of correction of Sep. 27, 1994.

Moxifloxacin

Moxifloxacin and its pharmaceutically acceptable salts, esters, and prodrugs thereof, can be used in the methods, compositions, and uses of the present invention. Moxifloxacin is sold under the tradename or proprietary Avelox. See U.S. Pat. No. 5,849,752, to Grunenberg et al., issued Dec. 15, 1998; U.S. Pat. No. 5,607,942, to Petersen et al., issued Mar. 4, 1997; and U.S. Pat. No. 4,990,517, to Petersen et al., issued Feb. 5, 1991 and its certificate of correction of Apr. 25, 1995.

Oritavancin

Oritavancin and its pharmaceutically acceptable salts, esters, and prodrugs thereof, can be used in the methods, compositions, and uses of the present invention. Oritavancin, which is a glycopeptide, is also known by the publicly disclosed code name LY333328. See R. C. Mercier et al., “Pharmacodynamic Evaluation of a New Glycopeptide, LY333328, and In Vitro Activity against Staphylococcus aureus and Enterococcus faecium”, Antimicrobial Agents and Chemotherapy, vol. 41, no. 6, pp. 1307-1312 (June 1997); U.S. Pat. No. 5,998,581, to Berglund et al., issued Dec. 7, 1999 and its certificate of correction of Nov. 14, 2000; U.S. Pat. No. 5,994,297, to Nicas et al., issued Nov. 30, 1999; U.S. Pat. No. 5,977,062, to Cooper et al., issued Nov. 2, 1999; U.S. Pat. No. 5,952,466, to Berglund et al, issued Sep. 14, 1999; U.S. Pat. No. 5,939,382, to Berglund et al., issued Aug. 17, 1999; U.S. Pat. No. 5,843,889, to Cooper et al., issued Dec. 1, 1998 and its certificate of correction of Mar. 28, 2000; U.S. Pat. No. 5,840,684, to Cooper et al., issued Nov. 24, 1998; PCT Application No. WO 00/66144, to Eli Lilly and Company, published Nov. 9, 2000; PCT Application No. WO 99/10006, to Eli Lilly and Company, published Mar. 4, 1999; PCT Application No. WO 98/22121, to Eli Lilly and Company, published May 28, 1998; PCT Application No. WO 98/21952, to Eli Lilly and Company, published May 28, 1998; and PCT Application No. WO 96/30401, to Eli Lilly and Company, published Oct. 3, 1996.

Oxazolidinones

Oxazolidinones and their pharmaceutically acceptable salts, esters, and prodrugs thereof, can be used in the methods, compositions, and uses of the present invention. Linezolid, i.e. (N-[[(5S)-3-[3-fluoro-4-(4-morpholinyl) phenyl]-2-oxo-5-oxazolidinyl]methyl]acetamide), which is sold under the tradename or proprietary name Zyvoxx, is a commercially marketed oxazolidinone. See U.S. Pat. No. 6,559,305 B1, to Bergren, issued May 6, 2003; U.S. Pat. No. 5,688,792, to Barbachyn et al., issued Nov. 18, 1997; and M.R. Barbychan et al., “Development of Linezolid: Oxazolidinone Structure-Activity Relationships Leading to Linezolid”, Angew. Chem. Int. Ed., 42, pp. 2010-2023 (2003). Other oxazolidinones and other compounds useful in the methods, compositions, and uses of the present invention are described in U.S. Pat. No. 6,969,726 B2, to Lou et al., issued Nov. 29, 2005; PCT Application No. WO 2006/022794, to Rib-X Pharmaceuticals, Inc., published Mar. 2, 2006; PCT Application No. WO 2005/070904, to Rib-X Pharmaceuticals, Inc., published Aug. 4, 2005; PCT Application No. WO 2005/061468, to Rib-X Pharmaceuticals, Inc., published Jul. 7, 2005; PCT Application No. WO 2005/019211, to Rib-X Pharmaceuticals, Inc., published Mar. 3, 2005; PCT Application No. WO 2005/012271, to Rib-X Pharmaceuticals, Inc., published Feb. 10, 2005; PCT Application No. WO 2005/012270, to Rib-X Pharmaceuticals, Inc., published Feb. 10, 2005; U.S. Patent Application Publication No. US 2005/0043317 A1, to Zhou et al., published Feb. 24, 2005; U.S. Patent Application Publication No. US 2005/0153971 A1, to Chen et al., published Jul. 14, 2005; U.S. Pat. No. 5,654,435 to Barbachyn et al., issued Aug. 5, 1997 and, PCT Application No. WO 2001/094342, to Dong A Pharm. Co., Ltd., published Dec. 13, 2001, and PCT Application No., WO 01/081350, to AstraZeneca AB and AstraZeneca UK Limited, published Nov. 1, 2001.

Other nonlimiting examples of oxazolidiones include those selected from the group consisting of the following compounds

| | |
|---|---|
| A | 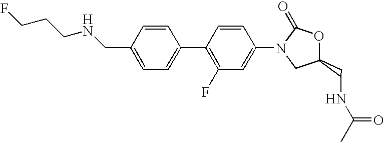 |
| | (5S)-N-(3-{2-Fluoro-4′-[(3-fluoro-propylamino)-methyl]-biphenyl-4- |
| | yl}-2-oxo-oxazolidin-5-ylmethyl)-acetamide |
| B | 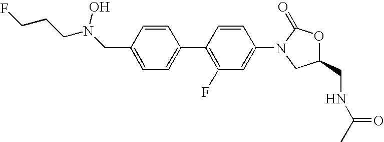 |
| | (5S)-N-[3 -(2-Fluoro-4′-{[(3-fluoro-propyl)-hydroxy-amino]- |
| | methyl}-biphenyl-4-yl)-2-oxo-oxazolidin-5-ylmethyl]-acetamide |
| C | 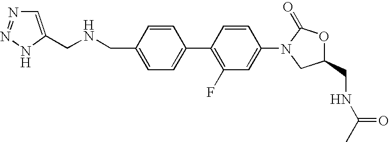 |
| | N-[3-(2-Fluoro-4′-{[(3H-[1,2,3]triazol-4-ylmethyl)-amino]-methyl}- |
| | biphenyl-4-yl)-2-oxo-oxazolidin-5-(S)-ylmethyl]-acetamide |
| D | 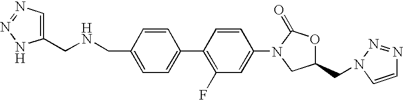 |
| | 3-(2-Fluoro-4′-{[(3H-[1,2,3]triazol-4-ylmethyl)-amino]-methyl}- |
| | biphenyl-4-yl)-5-(R)-[1,2,3]triazol-1-ylmethyl-oxazolidin-2-one |

or a pharmaceutically acceptable salt, ester, or prodrug thereof. An example of a salt would be the monohydrochloride salt of the four foregoing oxazolidinones A, B, C, and D.
Televancin

Televancin and its pharmaceutically acceptable salts, esters, and prodrugs thereof, can be used in the methods, compositions, and uses of the present invention. Televancin, which is a peptidoglycan, can be prepared by the sequential reduction amination of vancomycin and reaction with aminomehtylphosphonic acid. Televancin can also be prepared by the reductive alkylation of vancomycin with N-decyl-N-fluoroenyl-methyloxycarbonyl-2-aminoacetaldehyde via sodium cyano-borohydride and trifluoroacetic acid, and modification of the resorcinol position via Mannich aminomethylation. Televancin can also be prepared from vancomycin or its analogues by the sequential reaction with a protected amino-aldehyde, an amine and then an aminoalkylphosphonic acid in the presence of formaldehyde. See U.S. Pat. No. 6,887,976 B2, to Leadbetter et al., issued May 3, 2005; U.S. Pat. No. 6,878,686 B2, to Marquess et al., issued Apr. 12, 2005; U.S. Pat. No. 6,872,804 B2, to Mu, issued Mar. 29, 2005; U.S. Pat. No. 6,872,701 B2, to Leadbetter et al., issued Mar. 29, 2005; U.S. Pat. No. 6,858,584 B2, to Judice et al., issued Feb. 22, 2005; U.S. Pat. No. 6,831,150 B2, to Linsell, issued Dec. 14, 2004; U.S. Pat. No. 6,828,299 B2, to Yang et al., issued Dec. 7, 2004; U.S. Patent 6,770,621 B2, to Linsell et al., issued Aug. 3, 2004; U.S. Pat. No. 6,635,618 B2, to Leadbetter et al., issued Oct. 21, 2003; U.S. Pat. No. 6,620,781 B2, to Linsell et al., issued Sep. 16, 2003; U.S. Pat. No. 6,518,242 B1, to Chen et al. issued Feb. 11, 2003; and U.S. Pat. No. 6,455,669 B1, to Judice et al., issued Sep. 24, 2002; and PCT Application No. WO 03/029270, to Theravance, Inc., published Apr. 10, 2003.

DK-507k

The compound DK-507k and its pharmaceutically acceptable salts, esters, and prodrugs thereof, can be used in the methods, compositions, and uses of the present invention. DK-507k can be described as a fluoroquinolone. DK-507k is also known by the chemical name (−)-7-[(7S)-7-amino-5-azaspiro[2.4]heptan-5-yl]-6-fluoro-1-[(1R, 2S)-2-fluoro-1-cyclopropyl]-1,4-dihydro-8-methoxy-4-oxo-3-quinolinecarboxylic acid monohydrochloride monohydrate. See Otani et al., In Vitro and In Vivo antibacterial Activities ofDK-507k, a Novel Fluoroquinolone, Antimicrobial Agents and Chemotherapy, Vol. 47, no. 12, pages 3750-3759 (2003); Japanese Pat. No. JP 2004244380 A2, to Daiichi Seiyaku Co., Ltd., Japan, Sep. 2, 2004; PCT Application No. WO 2004/058261, to Daiichi Pharmaceutical Co., Ltd., Japan, published Jul. 15, 2004; PCT Patent Application No., WO 2003/076248, to Daiichi PHarmaceuitcal Co., Ltd., Japan, published Sep. 18, 2003; Japanese Pat. No. JP 2003096075 A2, to Daiichi Seiyaku Co., Ltd., Japan, Apr. 3, 2003; Japanese Pat. No. JP 2002255962 A2, to Daiichi Seiyaku Co., Ltd., Japan, Sep. 11, 2002; Japanese Pat. No. JP 2002201191 A2 to Daiichi Seiyaku Co., Ltd., Japan, Jul. 16, 2002; PCT Application No. WO 2001/072738, to Daiichi Pharmaceutical Co., Ltd., Japan, published Oct. 4, 2001; U.S. Pat. No. 6,900,225 B2, to Takemura et al., issued May 31, 2005; U.S. Patent Application No. 2004/142957 A1, to Takemura et al., published Jul. 22, 2004; U.S. Patent Application No. 2003/187008 A1, to Takemura et al., published Oct. 2, 2003; PCT Application No. WO 2001/058876, to Daiichi Pharmaceutical Co., Ltd., Japan, published Aug. 16, 2001; and U.S. Patent Application No. 2003/119848 A1, to Takemura et al., published Jun. 26, 2003.

DK-507k can be represented by the following formula;

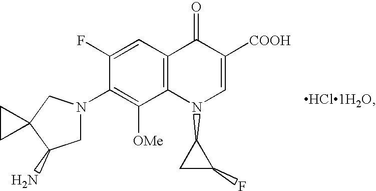

compound can also be obtained as crystals exhibiting characteristic peaks in the vicinity of angles of diffraction (2θ) of 6.9, 10.5, 14.4, 23.1, 26.9, and 27.8(°) when subjected to powder X-ray diffractometry.

The anhydrous free acid of the above compound, as well as other salts, esters, and prodrugs, and also hydrates of the compounds can be prepared and used in the present invention. Also other crystal forms of the foregoing can be prepared and used in the present invention.

Other Aspects of the Compounds of the Present Invention

Compounds designed, selected and/or optimized for use in the present invention, after being produced, can be characterized using a variety of assays known to those skilled in the art to determine whether the compounds have biological activity. For example, the compounds can be characterized by conventional assays, including but not limited to those assays described below, to determine whether the compounds have a predicted activity, binding activity and/or binding specificity.

Furthermore, high-throughput screening can be used to speed up analysis using such assays. As a result, it can be possible to screen rapidly the molecules described herein for activity, for example, as anti-cancer, anti-bacterial, anti-fungal, anti-parasitic or anti-viral agents. Also, it can be possible to assay how the compounds interact with a ribosome or ribosomal subunit and/or are effective as modulators (for example, inhibitors) of protein synthesis using techniques known in the art. General methodologies for performing high-throughput screening are described, for example, in Devlin, High Throughput Screening, (Marcel Dekker, 1998); and U.S. Pat. No. 5,763,263. High-throughput assays can use one or more different assay techniques including, but not limited to, those described below.

(1) Surface Binding Studies. A variety of binding assays can be useful in screening new molecules for their binding activity. One approach includes surface plasmon resonance (SPR) that can be used to evaluate the binding properties of molecules of interest with respect to a ribosome, ribosomal subunit or a fragment thereof.

SPR methodologies measure the interaction between two or more macromolecules in real-time through the generation of a quantum-mechanical surface plasmon. One device, (BIAcore Biosensor RTM from Pharmacia Biosensor, Piscatawy, N.J.) provides a focused beam of polychromatic light to the interface between a gold film (provided as a disposable biosensor “chip”) and a buffer compartment that can be regulated by the user. A 100 nm thick “hydrogel” composed of carboxylated dextran that provides a matrix for the covalent immobilization of analytes of interest is attached to the gold film. When the focused light interacts with the free electron cloud of the gold film, plasmon resonance is enhanced. The resulting reflected light is spectrally depleted in wavelengths that optimally evolved the resonance. By separating the reflected polychromatic light into its component wavelengths (by means of a prism), and determining the frequencies that are depleted, the BIAcore establishes an optical interface which accurately reports the behavior of the generated surface plasmon resonance. When designed as above, the plasmon resonance (and thus the depletion spectrum) is sensitive to mass in the evanescent field (which corresponds roughly to the thickness of the hydrogel). If one component of an interacting pair is immobilized to the hydrogel, and the interacting partner is provided through the buffer compartment, the interaction between the two components can be measured in real time based on the accumulation of mass in the evanescent field and its corresponding effects of the plasmon resonance as measured by the depletion spectrum. This system permits rapid and sensitive real-time measurement of the molecular interactions without the need to label either component.

(2) Fluorescence Polarization. Fluorescence polarization (FP) is a measurement technique that can readily be applied to protein-protein, protein-ligand, or RNA-ligand interactions in order to derive IC₅₀s and Kds of the association reaction between two molecules. In this technique one of the molecules of interest is conjugated with a fluorophore. This is generally the smaller molecule in the system (in this case, the compound of interest). The sample mixture, containing both the ligand-probe conjugate and the ribosome, ribosomal subunit or fragment thereof, is excited with vertically polarized light. Light is absorbed by the probe fluorophores, and re-emitted a short time later. The degree of polarization of the emitted light is measured. Polarization of the emitted light is dependent on several factors, but most importantly on viscosity of the solution and on the apparent molecular weight of the fluorophore. With proper controls, changes in the degree of polarization of the emitted light depends only on changes in the apparent molecular weight of the fluorophore, which in-turn depends on whether the probe-ligand conjugate is free in solution, or is bound to a receptor. Binding assays based on FP have a number of important advantages, including the measurement of IC₅₀s and Kds under true homogenous equilibrium conditions, speed of analysis and amenity to automation, and ability to screen in cloudy suspensions and colored solutions.

(3) Protein Synthesis. It is contemplated that, in addition to characterization by the foregoing biochemical assays, the compound of interest can also be characterized as a modulator (for example, an inhibitor of protein synthesis) of the functional activity of the ribosome or ribosomal subunit.

Furthermore, more specific protein synthesis inhibition assays can be performed by administering the compound to a whole organism, tissue, organ, organelle, cell, a cellular or subcellular extract, or a purified ribosome preparation and observing its pharmacological and inhibitory properties by determining, for example, its inhibition constant (IC₅₀) for inhibiting protein synthesis. Incorporation of ³H leucine or ³⁵S methionine, or similar experiments can be performed to investigate protein synthesis activity. A change in the amount or the rate of protein synthesis in the cell in the presence of a molecule of interest indicates that the molecule is a modulator of protein synthesis. A decrease in the rate or the amount of protein synthesis indicates that the molecule is a inhibitor of protein synthesis.

Furthermore, the compounds can be assayed for anti-proliferative or anti-infective properties on a cellular level. For example, where the target organism is a microorganism, the activity of compounds of interest can be assayed by growing the microorganisms of interest in media either containing or lacking the compound. Growth inhibition can be indicative that the molecule could be acting as a protein synthesis inhibitor. More specifically, the activity of the compounds of interest against bacterial pathogens can be demonstrated by the ability of the compound to inhibit growth of defined strains of human pathogens. For this purpose, a panel of bacterial strains can be assembled to include a variety of target pathogenic species, some containing resistance mechanisms that have been characterized. Use of such a panel of organisms permits the determination of structure-activity relationships not only in regards to potency and spectrum, but also with a view to obviating resistance mechanisms. The assays can be performed in microtiter trays according to conventional methodologies as published by The National Committee for Clinical Laboratory Standards (NCCLS) guidelines (NCCLS. M7-A5-Methods for Dilution Antimicrobial Susceptibility Tests for Bacteria That Grow Aerobically; Approved Standard-Fifth Edition. NCCLS Document M100-S12/M7 (ISBN 1-56238-394-9)).

4. Formulation and Administration

The methods of the present invention can be practiced by delivering the compounds of the present invention using any suitable carrier. The dose of active compound, mode of administration and use of suitable carrier will depend upon the intended patient or subject and the targeted microorganism, e.g., the target bacterial organism. The formulations, both for human medical use and veterinary use, of compounds according to the present invention typically include such compounds in association with a pharmaceutically acceptable carrier.

The carrier(s) should be “acceptable” in the sense of being compatible with compounds of the present invention and not deleterious to the recipient. Pharmaceutically acceptable carriers, in this regard, are intended to include any and all solvents, dispersion media, coatings, absorption delaying agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds (identified or designed according to the invention and/or known in the art) also can be incorporated into the compositions. The formulations can conveniently be presented in dosage unit form and can be prepared by any of the methods well known in the art of pharmacy/microbiology. In general, some formulations are prepared by bringing the compound into association with a liquid carrier or a finely divided solid carrier or both, and then, if necessary, shaping the product into the desired formulation.

A pharmaceutical composition of the invention should be formulated to be compatible with its intended route of administration. Solutions or suspensions can include the following components: a sterile diluent such as water, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide.

A wide variety of formulations and administration methods, including, e.g., intravenous formulations and administration methods can be found in S.K. Niazi, ed., Handbook of Pharmaceutical Formulations, Vols. 1-6 [Vol. 1 Compressed Solid Products, Vol. 2 Uncompressed Drug Products, Vol. 3 Liquid Products, Vol. 4 Semi-Solid Products, Vol. 5 Over the Counter Products, and Vol. 6 Sterile Products], CRC Press, Apr. 27, 2004.

Useful solutions for oral or parenteral administration can be prepared by any of the methods well known in the pharmaceutical art, described, for example, in Remington's Pharmaceutical Sciences, 18th ed. (Mack Publishing Company, 1990). Formulations for parenteral administration can also include glycocholate for buccal administration, methoxysalicylate for rectal administration, or citric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions which are solid at room temperature and liquid at body temperatures. Formulations also can include, for example, polyalkylene glycols such as polyethylene glycol, oils of vegetable origin, and hydrogenated naphthalenes. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration can be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug can also be administered in the form of a bolus, electuary or paste. A tablet can be made by compressing or molding the drug optionally with one or more accessory ingredients. Compressed tablets can be prepared by compressing, in a suitable machine, the drug in a free-flowing form such as a powder or granules, optionally mixed by a binder, lubricant, inert diluent, surface active or dispersing agent. Molded tablets can be made by molding, in a suitable machine, a mixture of the powdered drug and suitable carrier moistened with an inert liquid diluent.

Oral compositions generally include an inert diluent or an edible carrier. For the purpose of oral therapeutic administration, the active compound can be incorporated with excipients. Oral compositions prepared using a fluid carrier for use as a mouthwash include the compound in the fluid carrier and are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. Useful are carriers capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations. In the case of self-propelling solution and spray formulations, the effect can be achieved either by choice of a valve having the desired spray characteristics (i.e., being capable of producing a spray having the desired particle size) or by incorporating the active ingredient as a suspended powder in controlled particle size. For administration by inhalation, the compounds also can be delivered in the form of an aerosol spray from pressured container or dispenser which contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where adhesion to a tissue surface is desired the composition can include the drug dispersed in a fibrinogen-thrombin composition or other bioadhesive. The compound then can be painted, sprayed or otherwise applied to the desired tissue surface. Alternatively, the drugs can be formulated for parenteral or oral administration to humans or other mammals, for example, in effective amounts, e.g., amounts that provide appropriate concentrations of the drug to target tissue for a time sufficient to induce the desired effect.

Where the active compound is to be used as part of a transplant procedure, it can be provided to the living tissue or organ to be transplanted prior to removal of tissue or organ from the donor. The compound can be provided to the donor host. Alternatively or, in addition, once removed from the donor, the organ or living tissue can be placed in a preservation solution containing the active compound. In all cases, the active compound can be administered directly to the desired tissue, as by injection to the tissue, or it can be provided systemically, either by oral or parenteral administration, using any of the methods and formulations described herein and/or known in the art. Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution.

In conjunction with the methods of the present invention, pharmacogenomics (i.e., the study of the relationship between an individual's genotype and that individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a drug as well as tailoring the dosage and/or therapeutic regimen of treatment with the drug.

Generally, an effective amount of dosage of active compound will be in the range of from about 0.1 to about 100 mg/kg of body weight/day, more preferably from about 1.0 to about 50 mg/kg of body weight/day. The amount administered will also likely depend on such variables as the type of surgery or invasive medical procedure, the overall health status of the patient, the relative biological efficacy of the compound delivered, the formulation of the drug, the presence and types of excipients in the formulation, and the route of administration. Also, it is to be understood that the initial dosage administered can be increased beyond the above upper level in order to rapidly achieve the desired blood-level or tissue level, or the initial dosage can be smaller than the optimum.

Nolimiting doses of active compound comprise from about about 0.1 to about 1500 mg per dose. Nonlimitng examples of doses, which can be formulated as a unit dose for convenient administration to a patient include: about 25 mg, about 50 mg, about 75 mg, about 100 mg, about 125 mg, about 150 mg, about 175 mg, about 200 mg, about 225 mg, about 250 mg, about 275 mg, about 300 mg, about 325, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050, mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, and about 1500 mg. The foregoing doses are useful for administering the compounds of the present invention according to the methods of the present invention. The foregoing doses are particularly useful for administering the pyridone carboxylic acid compounds of the present invention, particularly the compound known by the names ABT-492 and WQ 3034 and also by the chemical name 1-(6-amino-3,5-difluoro-2-pyridinyl)-8-chloro-6-fluoro-1,4-dihydro-7-(3-hydroxy-1-azetidinyl)-4-oxo-3-quinolinecarboxylic acid, and pharmaceutically acceptable salts, esters and prodrugs thereof. The foregoing doses are also useful for administering D-glucitol-1-(6-amino-3,5-difluoro-2-pyridinyl)-8-chloro-6-fluoro-1,4-dihydro-7- (3-hydroxy-1-zetidinyl)-4-oxo-3-quinolinecarboxylic acid, and esters and prodrugs thereof

The compounds of the present invention and the dosees disclosed in-the previous paragraph can be administered to the patient from about 24 hours prior to up to immediately before the surgical or invasive medical procedure. Other times of administration are from about 12 hours prior to up to immediately before the surgical or invasive medical procedure, from about 11 hours prior to up to immediately before the surgical or invasive medical procedure, from about 10 hours prior to up to immediately before the surgical or invasive medical procedure, from about 9 hours prior to up to immediately before the surgical or invasive medical procedure, from about 8 hours prior to up to immediately before the surgical or invasive medical procedure, from about 7 hours prior to up to immediately before the surgical or invasive medical procedure, from about 6 hours prior to up to immediately before the surgical or invasive medical procedure, from about 5.5 hours prior to up to immediately before the surgical or invasive medical procedure, from about 5 hours prior to up to immediately before the surgical or invasive medical procedure, from about 4.5 hours prior to up to immediately before the surgical or invasive medical procedure, from about 4 hours prior to up to immediately before the surgical or invasive medical procedure, from about 3.5 hours prior to up to immediately before the surgical or invasive medical procedure, from about 3 hours prior to up to immediately before the surgical or invasive medical procedure, from about 2.5 hours prior to up to immediately before the surgical or invasive medical procedure, from about 2 hours prior to up to immediately before the surgical or invasive medical procedure, from about 1.5 hours prior to up to immediately before the surgical or invasive medical procedure, from about 1 hour prior to up to immediately before the surgical or invasive medical procedure, from about 30 minutes (0.5 hours) prior to up to immediately before the surgical or invasive medical procedure.

As is understood by one of ordinary skill in the art, generally, when dosages are described for a pharmaceutical active, the dosage is given on the basis of the parent or active moiety. Therefore, if a salt, hydrate, or another form of the parent or active moiety is used, a corresponding adjustment in the weight of the compound is made, although the dose is still refered to on the basis of the parent or active moiety delivered. As a nonlimiting example, if the parent or active moiety of interest is a monocarboxylic acid having a molecular weight of 250, and if the monosodium salt of the acid is desired to be delivered to be delivered at the same dosage, then an adjustment is made recognizing that the monosodium salt would have a molecular weight of approximately 272 (i.e. minus 1H or 1.008 atomic mass units and plus 1 Na or 22.99 atomic mass units). Therefore, a 250 mg dosage of the parent or active compound would corresponde to about 272 mg of the monosodium salt, which would also deliver 250 mg of the parent or active compound. Said another way, about 272 mg of the monosodium salt would be equivalent to a 250 mg dosage of the parent or active compound.

Using ABT-492 as a nonlimiting example, an example of a composition useful in the methods of the present invention can be about 100 mg of 1-(6-amino-3,5-difluoro-2-pyridinyl)-8- chloro-6-fluoro-1,4-dihydro-7-(3-hydroxy-1-azetidinyl)-4-oxo-3-quinolinecarboxylic acid, or a pharmaceutically acceptable salt, ester or prodrugs thereof, for administration to a patient from about 1 hour prior to up to immediately before a surgical or invasive medical procedure.

See, e.g., PCT Application No. WO 2005/019211 A2, published, Mar. 3, 2005, which describes various aspects useful in the present invention.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name.

### Example I

I-A. Formulation for Intravenous Administration

| Ingredients | Amount | |
|---|---|---|
| Antimicrobial Compound | 0.1-1500 | total mg |
| Dextrose, USP | 50 | mg/ml |
| Sodium citrate, USP | 1.60-1.75 | mg/ml |
| Citric Acid, USP | 0.80-0.90 | mg/ml |

| | |
|---|---|
| Water, USP | q.s |

This formulation for intravenous administration is formulated by heating water for injection to about 60° C. Next the sodium citrate, citric acid and dextrose are added and stirred until dissolved. A solution or aqueous slurry of the antimicrobial compound is added to the previous mixture and stirred until dissolved. The mixture is cooled to 25° C. with stirring. The pH is measured and adjusted if necessary. Lastly the mixture is brought to the desired volume, if necessary, with water for injection. The mixture is filtered, filled into the desired container (vial, syringe, infusion container, etc.), over wrapped and terminally moist heat sterilized.

This formulation is useful for intravenous administration, either bolus or infusion, to a patient for reducing the risk of or preventing infection due to a surgical or invasive medical procedure to be performed upon the patient. The formulation can be administered just prior to or up to about 1 hour prior to the surgical or invasive medical procedure.

II-B. Formulation for Intravenous Administration

| Ingredients | Amount | |
|---|---|---|
| 1-(6-amino-3,5-difluoro-2-pyridinyl)-8- | 0.1-1500 | total mg |
| chloro-6-fluoro-1,4-dihydro-7-(3-hydroxy- | | |
| 1-azetidinyl)-4-oxo-3-quinolinecarboxylic | | |
| acid (or a pharmaceutically acceptable | | |
| salt thereof) | | |
| Dextrose, USP | 50 | mg/ml |
| Sodium citrate, USP | 1.60-1.75 | mg/ml |
| Citric Acid, USP | 0.80-0.90 | mg/ml |

| | |
|---|---|
| Water, USP | q.s |

This formulation for intravenous administration is formulated by heating water for injection to about 60° C. Next the sodium citrate, citric acid and dextrose are added and stirred until dissolved. A solution or aqueous slurry of 1-(6-amino-3,5-difluoro-2-pyridinyl)-8-chloro-6-fluoro-1,4-dihydro-7-(3-hydroxy-1-azetidinyl)-4-oxo-3-quinolinecarboxylic acid (or a pharmaceutically acceptable salt thereof) is added to the previous mixture and stirred until dissolved. The mixture is cooled to 25° C. with stirring. The pH is measured and adjusted if necessary. Lastly the mixture is brought to the desired volume, if necessary with water for injection. The mixture is filtered, filled into the desired container (vials, syringe, infusion container, etc.), over wrapped and terminally moist heat sterilized.

This formulation is useful for intravenous administration, either bolus or infusion, to a patient for reducing the risk of or preventing infection due to a surgical or invasive medical procedure to be performed upon the patient. The formulation can be administered just prior to or up to about 1 hour prior to the surgical or invasive medical procedure.

### Example II

Alternatively, if the antimicrobial compound is prone to hydrolysis or a compact and convenient form to store formulation is desired, the antimicrobial compound can be provided as a lyophilisate which can be reconstituted before intravenous or intramuscular administration.

II-A. Lyophilisate for Reconstitution for Intravenous Administration

| Ingredient | mg per injection vial |
|---|---|
| Antimicrobial Compound | 0.1-1500 |
| Cyclodextin | 1500 |

Reconstitution solution for a volume to be administered of 50 ml (infusion): 5% aqueous glucose solution.

Reconstitution solution for a volume to be administered of 15 ml (bolus): 3.3% aqueous glucose solution.

The foregoing lyophilisate is useful for reconstitution and intravenous administration, either bolus or infusion, to a patient for reducing the risk of or preventing infection due to a surgical or invasive medical procedure to be performed upon the patient. The formulations can be administered just prior to or up to about 1 hour prior to the surgical or invasive medical procedure.

II-B Lyophilisate for Reconstitution for Intravenous Administration

| Ingredient | mg per injection vial |
|---|---|
| Antimicrobial Compound | 0.1-1500 |
| soya lecithin | 2250 |
| Sodium cholate | 1500 |

Reconstitution solution for a volume to be administered of 50 ml (infusion): 4% aqueous glucose solution.

Reconstitution solution for a volume to be administered of 15 ml (bolus): 2% aqueous glucose solution

The foregoing lyophilisate is useful for reconstitution and intravenous administration, either bolus or infusion, to a patient for reducing the risk of or preventing infection due to a surgical or invasive medical procedure to be performed upon the patient. The formulation can be administered just prior to or up to about 1 hour prior to the surgical or invasive medical procedure.

II-C Lyophilisate for Reconstitution for Intravenous Administration

| Ingredient | mg per injection vial |
|---|---|
| Antimicrobial Compound | 0.1-1500 |
| soya lecithin | 900 |
| Sodium glycocholate | 540 |

Reconstitution solution for a volume to be administered of 15 ml (bolus): 3.3% aqueous glucose solution.

The foregoing lyophilisate is useful for reconstitution and intravenous administration, either bolus or infusion, to a patient for reducing the risk of or preventing infection due to a surgical or invasive medical procedure to be performed upon the patient. The formulation can be administered just prior to or up to about 1 hour prior to the surgical or invasive medical procedure.

II-D Lyophilisate for Reconstitution for Intravenous Administration

| | mg per |
|---|---|
| Ingredient | injection vial |
| 1-(6-amino-3,5-difluoro-2-pyridinyl)-8-chloro-6-fluoro-1, | 0.1-1500 |
| 4-dihydro-7-(3-hydroxy1-azetidinyl)-4-oxo-3- | |
| quinolinecarboxylic acid (or a pharmaceutically acceptable | |
| salt thereof) | |
| Cyclodextin | 1500 |

Reconstitution solution for a volume to be administered of 50 ml (infusion): 5% aqueous glucose solution.

Reconstitution solution for a volume to be administered of 15 ml (bolus): 3.3% aqueous glucose solution.

The foregoing lyophilisate is useful for reconstitution and intravenous administration, either bolus or infusion, to a patient for reducing the risk of or preventing infection due to a surgical or invasive medical procedure to be performed upon the patient. The formulation can be administered just prior to or up to about 1 hour prior to the surgical or invasive medical procedure.

II-E Lyophilisate for Reconstitution for Intravenous Administration

| | mg per |
|---|---|
| | injection |
| Ingredient | vial |
| 1-(6-amino-3,5-difluoro-2-pyridinyl)-8-chloro-6- | 0.1-1500 |
| fluoro-1,4-dihydro-7-(3-hydroxy-1-azetidinyl)-4- | |
| oxo-3-quinolinecarboxylic acid (or a | |
| pharmaceutically acceptable salt thereof) | |
| soya lecithin | 2250 |
| sodium cholate | 1500 |

Reconstitution solution for a volume to be administered of 50 ml (infusion): 4% aqueous glucose solution.

Reconstitution solution for a volume to be administered of 15 ml (bolus): 2% aqueous glucose solution.

The foregoing lyophilisate is useful for reconstitution and intravenous administration, either bolus or infusion, to a patient for reducing the risk of or preventing infection due to a surgical or invasive medical procedure to be performed upon the patient. The formulation can be administered just prior to or up to about 1 hour prior to the surgical or invasive medical procedure.

II-F Lyophilisate for Reconstitution for Intravenous Administration

| | mg per injection |
|---|---|
| Ingredient | vial |
| 1-(6-amino-3,5-difluoro-2-pyridinyl)-8-chloro-6- | 0.1-1500 |
| fluoro-1,4-dihydro-7-(3-hydroxy-1-azetidinyl)-4-oxo- | |
| 3-quinolinecarboxylic acid (or a pharmaceutically | |
| acceptable salt thereof) | |
| soya lecithin | 900 |
| sodium glycocholate | 540 |

Reconstitution solution for a volume to be administered of 15 ml (bolus): 3.3% aqueous glucose solution

The foregoing lyophilisates are useful for reconstitution and intravenous administration, either bolus or infusion, to a patient for reducing the risk of or preventing infection due to a surgical or invasive medical procedure to be performed upon the patient. The formulation can be administered just prior to or up to about 1 hour prior to the surgical or invasive medical procedure.

II-G. Lyophilisate for Reconstitution for Intramuscular Administration

| Ingredient | mg per injection vial |
|---|---|
| Antimicrobial Compound | 0.1-1500 |
| Cyclodextin | 1500 |

Reconstituted the vial with the desired amount of diluent containing 0.9% socium chloride solution, 5% destrose solution, bacteriostatic water +0.9% benzyl alcohol, and 1% lidocaine solution (without epinephrine).

The foregoing lyophilisate is useful for reconstitution and intramuscular administration, to a patient for reducing the risk of or preventing infection due to a surgical or invasive medical procedure to be performed upon the patient. The formulations can be administered just prior to or up to about 1 hour prior to the surgical or invasive medical procedure.

II-H Lyophilisate for Reconstitution for Intramuscular Administration

| | mg per injection |
|---|---|
| Ingredient | vial |
| 1-(6-amino-3,5-difluoro-2-pyridinyl)-8-chloro-6- | 0.1-1500 |
| fluoro-1,4-dihydro-7-(3-hydroxy-1-azetidinyl)-4-oxo- | |
| 3-quinolinecarboxylic acid (or a pharmaceutically | |
| acceptable salt thereof) | |
| Cyclodextin | 1500 |

Reconstituted the vial with the desired amount of diluent containing 0.9% socium chloride solution, 5% destrose solution, bacteriostatic water +0.9% benzyl alcohol, and 1% lidocaine solution (without epinephrine).

The foregoing lyophilisate is useful for reconstitution and intramuscular administration, to a patient for reducing the risk of or preventing infection due to a surgical or invasive medical procedure to be performed upon the patient. The formulations can be administered just prior to or up to about 1 hour prior to the surgical or invasive medical procedure.

### Example III

III-A. Tablets for Oral Administration

| Ingredients | Per Tablet | | Per 4000 Tablets | |
|---|---|---|---|---|
| Antimicrobial Compound | 0.1-1500 | mg | 0.4-6000 | g |
| Anhydrous Lactose, NF | 110.45 | mg | 441.8 | g |
| Microcrystaline | 80.0 | mg | 320.0 | g |
| Cellulose NF | | | | |
| Magnesium Stearate | 1.00 | mg | 4.0 | g |
| Impalpable Powder NF | | | | |
| Croscarmellose Sodium | 2.00 | mg | 8.0 | g |
| NF Type A | | | | |

The antimicrobial compound (any of the compounds equivalent to the desired delivery strength, e.g., 50 to 1500 mg per tablet) is premixed with ⅓ of the microcrystaline cellulose NF and ½ of the anhydrous lactose NF in a ribbon blender for 5 minutes at 20 RPM. To the premix is added the remaining 2/3 of the microcrystaline cellulose NF and the remaining ½ of the anhydrous lactose NF. This is blended for 10 minutes at 20 RPM. Crosscarmellose sodium is added to the blended powders and mixed for 5 minutes at 20 RPM. Finally the magnesium stearate is added to the mixture by passing through a 90 mesh screen and blended for an additional 5 minutes at 20 RPM. The lubricated mixture is compressed to provide tablets of 500 mg active ingredient.

These tablets are useful for oral administration to a patient for reducing the risk of or preventing infection due to a surgical or invasive medical procedure to be performed upon the patient. One or more such tablets can be administered from about 30 minutes to about 4 hours prior to the surgical or invasive medical procedure.

III-B. Tablets for Oral Administration

| Ingredients | Per Tablet | | Per 4000 Tablets | |
|---|---|---|---|---|
| 1-(6-amino-3,5-difluoro-2-pyridinyl)- | 0.1-1500 | mg | 0.4-6000 | g |
| 8-chloro-6-fluoro-1,4-dihydro-7-(3- | | | | |
| hydroxy-1-azetidinyl)-4-oxo-3- | | | | |
| quinolinecarboxylic acid | | | | |
| Antimicrobial Compound (or a | | | | |
| pharmaceutically acceptable salt | | | | |
| thereof) | | | | |
| Anhydrous Lactose, NF | 110.45 | mg | 441.8 | g |
| Microcrystaline | 80.0 | mg | 320.0 | g |
| Cellulose NF | | | | |
| Magnesium Stearate | 1.00 | mg | 4.0 | g |
| Impalpable Powder NF | | | | |
| Croscarmellose Sodium | 2.00 | mg | 8.0 | g |
| NF Type A | | | | |

Using the procedure described in Example II.A., tables containing the compound 1-(6-amino-3,5-difluoro-2-pyridinyl)-8-chloro-6-fluoro-1,4-dihydro-7-(3-hydroxy-1-azetidinyl)-4-oxo-3-quinolinecarboxylic acid (or a pharmaceutically acceptable salt thereof) are prepared.

These tablets are useful for oral administration to a patient for reducing the risk of or preventing infection due to a surgical or invasive medical procedure to be performed upon the patient. One or more such tablets can be administered from about 30 minutes to about 4 hours prior to the surgical or invasive medical procedure.

### INCORPORATION BY REFERENCE

The entire disclosure of each of the patent documents, including certificates of correction, patent application documents, scientific articles, governmental reports, websites, and other references referred to herein is incorporated by reference in its entirety for all purposes. In case of a conflict in terminology, the present specification controls.

### EQUIVALENTS

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of reducing the risk of a microbial infection in a patient having a surgical or invasive medical procedure comprising administering a prophylactically effective amount of an antimicrobial compound to said patient prior to said surgical or invasive procedure.

2. A method of preventing a microbial infection in a patient having a surgical or invasive medical procedure comprising administering a prophylactically effective amount of an antimicrobial compound to said patient prior to said surgical or invasive procedure.

3. A method of peri-operative prophylaxis in a patient in need thereof comprising administering a prophylactically effective amount of an antimicrobial compound to said patient prior to said patient undergoing a surgical or invasive medical procedure.

4. The method according to claim 1 wherein said microbial infection is a bacterial infection.

5. The method according to claim 1 wherein said patient is a human.

6. The method according to claim 1 wherein said compound is administered intravenously.

7. The method according to claim 1 wherein said compound is administered orally, subcutaneously, parenteraly, or intramuscularly.

8. The method according to claim I wherein said compound is administered between about 24 hours prior to said surgical or invasive procedure to immediately before said surgical or invasive procedure.

9. The method according to claim 1 wherein said antimicrobial agent is selected from a compound that binds to or modulates bacterial ribosomal RNA.

10. The method according to claim 1 wherein said antimicrobial agent is selected from a compound that binds to or modulates the large ribosomal subunit of a bacterial organism.

11. The method according to claim 1 wherein said antimicrobial agent is selected from macrolides, ketolides, streptogramin As, streptogramin Bs, chloramphenicol and chloramphenicol derivatives, fluorfenicol and fluorfenicol derivatives, glycopeptides, pleuromutilins, aminoglycosides, beta-lactams and carbapenems (including carbapenems with a 7-acylated imidazo[5- l ,b]thiazole-2-yl group directly attached to the carbapenem moiety of the C-2 position), cephalosporins, lincosamides, quinolones, fluoroquinolones, pyridonecarboxylic acid derivatives, benzoheterocyclic compounds, aminomethylcycline compounds, dalbavancin, daptomycin, garenoxacin, gatifloxacin, gemifloxacin, levofloxacin, moxifloxacin, oritavancin, oxazolidinones (e.g., linezolid and other oxazolidinones), televancin, DK-507k [also known as, (−)-7-[(7S)-7-amino-5-azaspiro[2.4]heptan-5-yl]-6-fluoro-1-[(1R, 2S)-2-fluoro-1-cyclopropyl]-1,4-dihydro-8-methoxy-4-oxo-3-quinolinecarboxylic acid monohydrochloride monohydrate],and mixtures thereof.

12. The method according to claim 11 wherein said compound is a pyridonecarboxylic acid derivative.

13. The method according to claim 12 wherein said compound is selected from a pyridonecarboxylic acid derivative corresponding to the following structure:
Pyridonecarboxylic Acid Derivative I 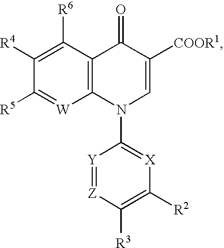
wherein R¹ represents a hydrogen atom or a carboxyl protective group; R² represents a hydroxyl group, a lower alkoxy group, or a substituted or unsubstituted amino group; R³ represents a hydrogen atom or a halogen atom; R⁴ represents a hydrogen atom or a halogen atom; R⁵ represents a halogen atom or an optionally substituted saturated cyclic amino group; R represents a hydrogen atom, a halogen atom, a nitro group, or an optionally protected amino group; X, Y and Z may be the same or different and respectively represent a nitrogen atom, CH or CR⁷ (wherein R⁷ represents a lower alkyl group, a halogen atom, or a cyano group), with the proviso that at least one of X, Y and Z represent a nitrogen atom, and W represents a nitrogen atom or CR⁸ (wherein R⁸ represents a hydrogen atom, a halogen atom, or a lower alkyl group), and with the proviso that when R¹ represents a hydrogen atom, R² represents an amino group, R³ and R⁴ represent a fluorine atom, R⁶ represents a hydrogen atom, X represents a nitrogen atom, Y represents CR⁷ (wherein R⁷ represents a fluorine atom), Z represents CH, and W is CR⁸ (wherein R⁸represents a chlorine atom), then R⁵is not a 3-hydroxyazetidine-1-yl group;
or a pharmaceutically acceptable salt, ester, or prodrug thereof; with the proviso that R¹, R², R³, R⁴, R⁵, R⁶, R⁷, R⁸, W, X, Y, and Z are defined with respect to this claim 16 and any such claims on which this claim 16 depends.

14. The method according to claim 1 wherein said compound is selected from a pyridonecarboxylic acid corresponding to the following structure: 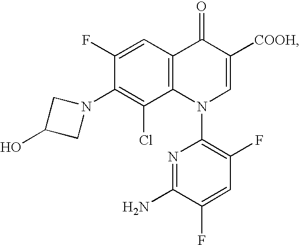
or a pharmaceutically acceptable salt, ester, or prodrug thereof.

15. The method of claim 1 wherein said compound is D-glucitol 1-(6-amino-3,5-difluoro-2-pyridinyl)-8-chloro-6-fluoro-1,4-dihydro-7-(3-hydroxy-1-azetidinyl)-4-oxo-3-quinolinecarboxylate (salt).

16. The method according to claim 15 wherein said compound is crystalline D-glucitol 1-(6-amino-3,5-difluoro-2-pyridinyl)-8-chloro-6-fluoro-1,4-dihydro-7-(3-hydroxy-1-azetidinyl)-4-oxo-3-quinolinecarboxylate (salt) characterized, when measured about about 25° C. with Cu—Ka radiation, by the powder diffraction pattern shown in FIG. 1.

17. The method according to claim 1 wherein said compound is D-glucitol 1-(6-amino-3,5-difluoro-2-pyridinyl)-8-chloro-6-fluoro-1,4-dihydro-7-(3-hydroxy-1-azetidinyl)-4-oxo-3-quinolinecarboxylate trihydrate (salt).

18. The method according to claim 17 wherein said compound is crystalline D-glucitol I-(6-amino-3,5-difluoro-2-pyridinyl)-8-chloro-6-fluoro-1,4-ihydro-7-(3-hydroxy-1-azetidinyl)-4-oxo-3-quinolinecarboxylate trihydrate (salt) characterized, when measured about about 25° C. with Cu-Ka radiation, by the powder diffraction pattern shown in FIG. 2.

19. The method according to claim 1, wherein said compound comprises from about 0.1 to about 1500 mg.

20. The method according to claim 19, wherein said compound comprises about 25 mg, or about 50 mg, or about 75 mg, or about 100 mg, or about 125 mg, or about 150 mg, or about 175 mg, or about 200 mg, or about 225 mg, or about 250 mg, or about 275 mg, or about 300 mg, or about 325, or about 350 mg, or about 375 mg, or about 400 mg, or about 425 mg, or about 450 mg, or about 475 mg, or about 500 mg, or about 525 mg, or about 550 mg, or about 575 mg, or about 600 mg, or about 625 mg, or about 650 mg, or about 675 mg, or about 700 mg, or about 725 mg, or about 750 mg, or about 775 mg, or about 800 mg, or about 825 mg, or about 850 mg, or about 875 mg, or about 900 mg, or about 925 mg, or about 950 mg, or about 975 mg, or about 1000 mg, or about 1025 mg, or about 1050, mg, or about 1075 mg, or about 1100 mg, or about 1125 mg, or about 1150 mg, or about 1175 mg, or about 1200 mg, or about 1225 mg, or about 1250 mg, or about 1275 mg, or about 1300 mg, or about 1325 mg, or about 1350 mg, or about 1375 mg, or about 1400 mg, or about 1425 mg, or about 1450 mg, or about 1475 mg, or about 1500 mg.

21. The method according to claim 1, wherein the compound is administered about 24 hours prior to, or about 20 hours prior to, or about 16 hours prior to, or about 12 hours prior to, or about 10 hours prior to, or about 8 hours prior to, or about 6 hours prior to, or about 4 hours prior to, or about 2 hours prior to, or about 1 hour prior to, or about 30 minutes prior to, or immediately prior to said surgical or invasive medical procedure.
